SEQUENCE LISTING (43 sequences)

SEQ ID NO: 1 - DNA, 5014 bp, Homo sapiens
gagtggagttctggaggaatgtttaccagacacagagcccagagggacagcgcccagagcccagatagagagacacggcctcactggctcagcaccagggtccccttccccctcctcagctccctccctggcccctttaagaaagagctgatcctctcctctcttgagttaacccctgattgtccaggtggcccctggctctggcctggtgggcggaggcaaagggggagccaggggcggagaaagggttgcccaagtctgggagtgagggaaggaggcaggggtgctgagaaggcggctgctgggcagagccggtggcaagggcctcccctgccgctgtgccaggcaggcagtgccaaatccggggagcctggagctggggggagggccggggacagcccggccctgccccctcccccgctgggagcccagcaacttctgaggaaagtttggcacccatggcgtggcggtgccccaggatgggcagggtcccgctggcctggtgcttggcgctgtgcggctgggcgtgcatggcccccaggggcacgcaggctgaagaaagtcccttcgtgggcaacccagggaatatcacaggtgcccggggactcacgggcacccttcggtgtcagctccaggttcagggagagccccccgaggtacattggcttcgggatggacagatcctggagctcgcggacagcacccagacccaggtgcccctgggtgaggatgaacaggatgactggatagtggtcagccagctcagaatcacctccctgcagctttccgacacgggacagtaccagtgtttggtgtttctgggacatcagaccttcgtgtcccagcctggctatgttgggctggagggcttgccttacttcctggaggagcccgaagacaggactgtggccgccaacacccccttcaacctgagctgccaagctcagggacccccagagcccgtggacctactctggctccaggatgctgtccccctggccacggctccaggtcacggcccccagcgcagcctgcatgttccagggctgaacaagacatcctctttctcctgcgaagcccataacgccaagggggtcaccacatcccgcacagccaccatcacagtgctcccccagcagccccgtaacctccacctggtctcccgccaacccacggagctggaggtggcttggactccaggcctgagcggcatctaccccctgacccactgcaccctgcaggctgtgctgtcagacgatgggatgggcatccaggcgggagaaccagaccccccagaggagcccctcacctcgcaagcatccgtgcccccccatcagcttcggctaggcagcctccatcctcacaccccttatcacatccgcgtggcatgcaccagcagccagggcccctcatcctggacccactggcttcctgtggagacgccggagggagtgcccctgggcccccctgagaacattagtgctacgcggaatgggagccaggccttcgtgcattggcaagagccccgggcgcccctgcagggtaccctgttagggtaccggctggcgtatcaaggccaggacaccccagaggtgctaatggacatagggctaaggcaagaggtgaccctggagctgcagggggacgggtctgtgtccaatctgacagtgtgtgtggcagcctacactgctgctggggatggaccctggagcctcccagtacccctggaggcctggcgcccagggcaagcacagccagtccaccagctggtgaaggaaccttcaactcctgccttctcgtggccctggtggtatgtactgctaggagcagtcgtggccgctgcctgtgtcctcatcttggctctcttccttgtccaccggcgaaagaaggagacccgttatggagaagtgtttgaaccaacagtggaaagaggtgaactggtagtcaggtaccgcgtgcgcaagtcctacagtcgtcggaccactgaagctaccttgaacagcctgggcatcagtgaagagctgaaggagaagctgcgggatgtgatggtggaccggcacaaggtggccctggggaagactctgggagagggagagtttggagctgtgatggaaggccagctcaaccaggacgactccatcctcaaggtggctgtgaagacgatgaagattgccatctgcacgaggtcagagctggaggatttcctgagtgaagcggtctgcatgaaggaatttgaccatcccaacgtcatgaggctcatcggtgtctgtttccagggttctgaacgagagagcttcccagcacctgtggtcatcttacctttcatgaaacatggagacctacacagcttcctcctctattcccggctcggggaccagccagtgtacctgcccactcagatgctagtgaagttcatggcagacatcgccagtggcatggagtatctgagtaccaagagattcatacaccgggacctggcggccaggaactgcatgctgaatgagaacatgtccgtgtgtgtggcggacttcgggctctccaagaagatctacaatggggactactaccgccagggacgtatcgccaagatgccagtcaagtggattgccattgagagtctagctgaccgtgtctacaccagcaagagcgatgtgtggtccttcggggtgacaatgtgggagattgccacaagaggccaaaccccatatccgggcgtggagaacagcgagatttatgactatctgcgccagggaaatcgcctgaagcagcctgcggactgtctggatggactgtatgccttgatgtcgcggtgctgggagctaaatccccaggaccggccaagttttacagagctgcgggaagatttggagaacacactgaaggccttgcctcctgcccaggagcctgacgaaatcctctatgtcaacatggatgagggtggaggttatcctgaaccccctggagctgcaggaggagctgaccccccaacccagccagaccctaaggattcctgtagctgcctcactgcggctgaggtccatcctgctggacgctatgtcctctgcccttccacaacccctagccccgctcagcctgctgataggggctccccagcagccccagggcaggaggatggtgcctgagacaaccctccacctggtactccctctcaggatccaagctaagcactgccactggggaaaactccaccttcccactttcccaccccacgccttatccccacttgcagccctgtcttcctacctatcccacctccatcccagacaggtccctccccttctctgtgcagtagcatcaccttgaaagcagtagcatcaccatctgtaaaaggaaggggttggattgcaatatctgaagccctcccaggtgttaacattccaagactctagagtccaaggtttaaagagtctagattcaaaggttctaggtttcaaagatgctgtgagtctttggttctaaggacctgaaattccaaagtctctaattctattaaagtgctaaggttctaaggcctacttttttttttttttttttttttttttttttttttgcgatagagtctcactgtgtcacccaggctggagtgcagtggtgcaatctcgcctcactgcaaccttcacctaccgagttcaagtgattttcctgccttggcctcccaagtagctgggattacaggtgtgtgccaccacacccggctaatttttatatttttagtagagacagggtttcaccatgttggccaggctggtctaaaactcctgacctcaagtgatctgcccacctcagcctcccaaagtgctgagattacaggcatgagccactgcactcaaccttaagacctactgttctaaagctctgacattatgtggttttagattttctggttctaacatttttgataaagcctcaaggttttaggttctaaagttctaagattctgattttaggagctaaggctctatgagtctagatgtttattcttctagagttcagagtccttaaaatgtaagattatagattctaaagattctatagttctagacatggaggttctaaggcctaggattctaaaatgtgatgttctaaggctctgagagtctagattctctggctgtaaggctctagatcataaggcttcaaaatgttatcttctcaagttctaagattctaatgatgatcaattatagtttctgaggctttatgataatagattctcttgtataagatcctagatcctaagggtcgaaagctctagaatctgcaattcaaaagttccaagagtctaaagatggagtttctaaggtccggtgttctaagatgtgatattctaagacttactctaagatcttagattctctgtgtctaagattctagatcagatgctccaagattctagatgattaaataagattctaacggtctgttctgtttcaaggcactctagattccattggtccaagattccggatcctaagcatctaagttataagactctcacactcagttgtgactaactagacaccaaagttctaataatttctaatgttggacacctttaggttctttgctgcattctgcctctctaggaccatggttaagagtccaagaatccacatttctaaaatcttatagttctaggcactgtagttctaagactcaaatgttctaagtttctaagattctaaaggtccacaggtctagactattaggtgcaatttcaaggttctaaccctatactgtagtattctttggggtgcccctctccttcttagctatcattgcttcctcctccccaactgtgggggtgtgcccccttcaagcctgtgcaatgcattagggatgcctcctttcccgcaggggatggacgatctcccacctttcgggccatgttgcccccgtgagccaatccctcaccttctgagtacagagtgtggactctggtgcctccagaggggctcaggtcacataaaactttgtatatcaacgaaaaaaa

SEQ ID NO: 2 - protein, 894 aa, Homo sapiens
MAWRCPRMGRVPLAWCLALCGWACMAPRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLVKEPSTPAFSWPWWYVLLGAVVAAACVLILALFLVHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFTELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 3 - RNA, 21 nt, Artificial
  Axl-specific siRNA
ggaaagaggugaacugguauu

SEQ ID NO: 4 - RNA, 21 nt, Artificial
  Axl-specific siRNA
caagaugaauggaaaguuguu

SEQ ID NO: 5 - RNA, 21 nt, Artificial
  Axl-specific siRNA
ggaacugcaugcugaaugauu

SEQ ID NO: 6 - RNA, 21 nt, Artificial
  Axl-specific siRNA
ggaagaaggagacucgauauu

SEQ ID NO: 7 - RNA, 19 nt, Artificial
  control siRNA
gguagcuauucaguuacug

SEQ ID NO: 8 - RNA, 20 nt, Artificial
  Runx2/Cbfa1-specific siRNA
cgugaauggucauaauaacu

SEQ ID NO: 9 - DNA, 19 bp, Artificial
  Oligonucleotide primer used to detect osteocalcin
cggccctgagtctgacaaa

SEQ ID NO: 10 - DNA, 22 bp, Artificial
  Oligonucleotide primer used to detect osteocalcin
gccggagtctgttcactacctt

SEQ ID NO: 11 - DNA, 24 bp, Artificial
  Oligonucleotide primer used to detect osteocalcin
ccttcatgtccaagcaggagggca

SEQ ID NO: 12 - protein, 14 aa, Homo sapiens
VKEPSTPAFSWPWW

SEQ ID NO: 13 - protein, 429 aa, Homo sapiens
MGHHHHHHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFAELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 14 - protein, 21 aa, Homo sapiens
KQPADCLDGLYALMSRCWELN

SEQ ID NO: 15 - protein, 20 aa, Homo sapiens
FGLSKKIYNGDYYRQGRIAK

SEQ ID NO: 16 - protein, 13 aa, Homo sapiens
DCLDGLYALMSRC

SEQ ID NO: 17 - protein, 12 aa, Homo sapiens
KKIYNGDYYRQG

SEQ ID NO: 18 - DNA, 49 bp, Homo sapiens
cggacatcagaccttcgtgtcttcctgtcaacacgaaggtctgatgtcc

SEQ ID NO: 19 - DNA, 49 bp, Homo sapiens
cgcgtatcaaggccaggacacttcctgtcatgtcctggccttgatacgc

SEQ ID NO: 20 - DNA, 49 bp, Homo sapiens
cgagtgaagcggtctgcatgcttcctgtcacatgcagaccgcttcactc

SEQ ID NO: 21 - DNA, 49 bp, Homo sapiens
cgagtaccaagagattcatacttcctgtcatatgaatctcttggtactc

SEQ ID NO: 22 - DNA, 49 bp, Homo sapiens
cgacgaaatcctctatgtcacttcctgtcatgacatagaggatttcgtc

SEQ ID NO: 23 - DNA, 49 bp, Mus musculus
cggcttcgagatggacagatcttcctgtcaatctgtccatctcgaagcc

SEQ ID NO: 24 - DNA, 49 bp, Mus musculus
cgtaccggctggcatatcgacttcctgtcatcgatatgccagccggtac

SEQ ID NO: 25 - DNA, 49 bp, Mus musculus
cgtgtccgaaagtcctacagcttcctgtcactgtaggactttcggacac

SEQ ID NO: 26 - DNA, 49 bp, Mus musculus
cgaaacacggagacctacaccttcctgtcagtgtaggtctccgtgtttc

SEQ ID NO: 27 - DNA, 49 bp, Mus musculus
cgtcaaggaaatcggctgaacttcctgtcattcagccgatttccttgac

SEQ ID NO: 28 - RNA, 19 nt, Artificial
  Budagian et al. siRNA
uaucacaggugccagagga

SEQ ID NO: 29 - RNA, 21 nt, Artificial
  Budagian et al. siRNA
aagacauccucuuucuccugc

SEQ ID NO: 30 - RNA, 21 nt, Artificial
  Budagian et al. siRNA
aagauuuggagaacacacuga

SEQ ID NO: 31 - DNA, 64 bp, Artificial
  Holland et al shRNA
gacatcctctttctcctgcgaagcccatgaagcttgatgggcttcgcaggagaaagaggatgtc

SEQ ID NO: 32 - DNA, 64 bp, Artificial
  Holland et al. shRNA
gatttggagaacacactgaaggccttgcgaagcttggcaaggccttcagtgtgttctccaaatc

SEQ ID NO: 33 - protein, 84 aa, Homo sapiens
TLRCQLQVQGEPPEVHWLRDGQILELADSTQTQVPLGEDEQDDWIVVSQLRITSLQLSDTGQYQCLVFLGHQTFVSQPGYVGLE

SEQ ID NO: 34 - protein, 86 aa, Homo sapiens
GLPYFLEEPEDRTVAANTPFNLSCQAQGPPEPVDLLWLQDAVPLATAPGHGPQRSLHVPGLNKTSSFSCEAHNAKGVTTSRTATIT

SEQ ID NO: 35 - protein, 17 aa, Homo sapiens
AGAGGGTDEGIYDVPLL

SEQ ID NO: 36 - protein, 16 aa, Homo sapiens
AGAGGPQDIYDVPPVR

SEQ ID NO: 37 - protein, 429 aa, Homo sapiens
MGRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFAELREDLENTLKALPPAQEPDEILYVNMDEGGGYPEPPGAAGGADPPTQPDPKDSCSCLTAAEVHPAGRYVLCPSTTPSPAQPADRGSPAAPGQEDGAHHHHHH

SEQ ID NO: 38 - protein, 402 aa, Homo sapiens
ELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFAELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 39 - protein, 385 aa, Homo sapiens
EATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFAELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 40 - protein, 367 aa, Homo sapiens
DVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFAELREDLENTLKALPPAQEPDEILYVNMDEGGGYPEPPGAAGGADPPTQPDPKDSCSCLTAAEVHPAGRYVLCPSTTPSPAQPADRGSPAAPGQEDGA

SEQ ID NO: 41 - protein, 354 aa, Homo sapiens
MGRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFAELREDLENTLKALPPAQEPDEILYVNMD

SEQ ID NO: 42 - protein, 337 aa, Homo sapiens
MGRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFAELREDLENTLK

SEQ ID NO: 43 - protein, 430 aa, Homo sapiens
MHRRKKETRYGEVFEPTVERGELVVRYRVRKSYSRRTTEATLNSLGISEELKEKLRDVMVDRHKVALGKTLGEGEFGAVMEGQLNQDDSILKVAVKTMKIAICTRSELEDFLSEAVCMKEFDHPNVMRLIGVCFQGSERESFPAPVVILPFMKHGDLHSFLLYSRLGDQPVYLPTQMLVKFMADIASGMEYLSTKRFIHRDLAARNCMLNENMSVCVADFGLSKKIYNGDYYRQGRIAKMPVKWIAIESLADRVYTSKSDVWSFGVTMWEIATRGQTPYPGVENSEIYDYLRQGNRLKQPADCLDGLYALMSRCWELNPQDRPSFAELREDLENTLKALPPAQEPDEILYVNMDEGGGYPEPPGAAGGADPPTQPDPKDSCSCLTAAEVHPAGRYVLCPSTTPSPAQPADRGSPAAPGQEDGAGHHHHHH

(19) United States
(12) Patent Application Publication
(10) Pub. No.: US 2009/0087431 A1
(43) Pub. Date: Apr. 2, 2009

(54) METHODS OF TREATING BONE DISORDERS WITH MODULATORS OF AXL

(75) Inventors: Paul John YAWORSKY, Boston, MA (US); Erica Ann Smith, New London, CT (US); Michael John Cain, Exeter, NH (US); John Allen Robinson, Downingtown, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/166,464

(22) Filed: Jul. 2, 2008

Related U.S. Application Data
(60) Provisional application No. 60/958,316, filed on Jul. 3, 2007.
(60) Provisional application No. 60/958,270, filed on Jul. 2, 2007.

(51) Int. Cl. A61K 39/395; A61K 31/7105; A61K 38/16; A61P 19/08; C12Q 1/68; C07H 21/02

(52) U.S. Cl. 424/133.1; 424/130.1; 514/44; 514/12; 435/6; 536/23.1

(57) ABSTRACT

The invention provides methods for treating or preventing bone and cartilage disorders comprising administering to a mammal an inhibitor of Axl gene expression or an inhibitor of Axl protein activity.

### CROSS-REFERENCE TO RELATED APPLICATIONS

This application claims the benefit of U.S. Provisional Application No. 60/958,270, filed on Jul. 2, 2007 and U.S. Provisional Application No. 60/958,316, filed on Jul. 3, 2007. The entire content of these applications is hereby incorporated by reference in its entirety.

### FIELD OF THE INVENTION

The invention relates to therapeutic uses of Axl modulators in the treatment of bone disorders such as osteoporosis, osteopenia, osteomalacia, osteodystrophy, osteoarthritis, osteomyeloma, bone fracture, Paget's disease, osteogenesis imperfecta, bone sclerosis, aplastic bone disorder, humoral hypercalcemic myeloma, multiple myeloma, bone thinning following metastasis, hypercalcemia, chronic renal disease, kidney dialysis, primary hyperparathyroidism, secondary hyperparathyroidism, inflammatory bowel disease, Crohn's disease, long-term use of corticosteroids, or long-term use of gonadotropin releasing hormone (GnRH) agonists or antagonists.

### BACKGROUND OF THE INVENTION

Receptor tyrosine kinases (“RTKs”) are involved in the transduction of signals from the extracellular environment. Such signals induce a wide variety of cellular responses, including proliferation, differentiation, migration, and metabolism. Based on sequence similarity, known RTKs are classified into more than ten distinct subfamilies. The Mer receptor subfamily includes Axl, Tyro3, and Mer. Axl is also known as Ufo, Tyro7, and Ark; among others; and is referred to as “Axl” herein. Cloned as a novel Axl-homologous RTK, Tyro3 is also known as Rse, Brt, and Sky, among others; and is referred to as “Tyro3” herein. Named after its original reported expression pattern in humans (monocytes and epithelial and reproductive tissues), Mer is a putative mammalian homologue of chicken c-eyk. These receptors share a distinct structure characterized by an extracellular domain containing two immunoglobulin-like domains, two fibronectin type III repeats, a transmembrane domain, and a cytoplasmic domain that contains a conserved catalytic kinase region (Heiring et al., J. Biol. Chem. 279:6052-6058 (2004); Nagata et al., J. Biol. Chem. 271:30022-30027 (1996)).

A single ligand, growth arrest-specific gene 6 (Gas6), activates the tyrosine kinase activity of the Mer receptor subfamily (Varnum et al., Nature 373:623-626 (1995); Mark et al., J. Biol. Chem. 271:9785-9789 (1996); Chen et al., Oncogene 14:2033-2039 (1997)). Gas6 encodes a vitamin K-dependent protein that binds to Axl, Tyro3, and Mer with nanomolar affinities (Nagata et al., J. Biol. Chem. 271:9785-9789 (1996)). Axl, Mer, and Tyro3 triple knockout mice display a lymphoproliferation and autoimmune phenotype, but mice lacking only Axl have no immune phenotype, indicating that these three related kinases function in concert (Lu et al., Science 293:306-311 (2001)). Binding of Gas6 to Axl regulates cell adhesion, proliferation, and aggregation, and has been implicated in some cancers, including chronic myelogenous leukemia, colon cancer, and melanoma. A role for Axl has been suggested in a wide variety of physiological processes, including spermatogenesis, vascular cell function, progression of type 2 diabetes, and neural development. Axl has also been implicated in cardiovascular disorders, as it is expressed in pericytes, including during ectopic calcification associated with atherosclerotic lesions (Collett et al., Circ. Res. 92:1123-1129 (2003)).

Axl is expressed in bone marrow stromal cells, a population which contains osteoprogenitor cells (Satomura et al., J. Cell. Physiol. 177:426-438 (1998)). Axl expression has also been looked at in osteoprogenitor cell lines, where addition of bone morphogenetic protein 2 (BMP2) inhibited Axl mRNA expression (PCT Publication No. WO 02/081745; U.S. Patent Publication No. 20060030541). However, in spite of this observation, it remains unclear whether Axl plays any direct role during bone development, or whether the inhibition of Axl is merely a consequence of the activity of BMP2.

Osteoporosis, the most prevalent bone disorder in America, currently affects an estimated 20 million people, with another 34 million Americans having sufficiently low bone mass to place them at a heightened risk of osteoporosis in the future. Osteoporosis accounts for 1.5 million bone fractures every year, with roughly 85% of those fractures occurring in the patient's hip, spine, or wrist. Although osteoporosis affects both sexes, it is observed most frequently in postmenopausal women. Decreased bone mass and/or bone mineral density (BMD) may also result from chronic glucocorticoid therapy, premature gonadal failure, androgen suppression, vitamin D deficiency, insufficient calcium intake, secondary hyperparathyroidism, or anorexia nervosa.

Thus, there is a continuing need to develop new therapies for bone disorders, such as osteoporosis and osteoarthritis, especially for humans.

### SUMMARY OF THE INVENTION

In one embodiment, the invention provides a method of treating or preventing a bone disorder in a mammal comprising administering to the mammal an inhibitor of Axl gene expression or an inhibitor of Axl protein activity. In one embodiment, the invention provides a method wherein the inhibitor is not bone morphogenetic protein 2 (BMP2). In one embodiment, the inhibitor decreases the tyrosine kinase activity of Axl protein. In one embodiment, the invention provides a method wherein the inhibitor of Axl protein activity has the structural formula (I):

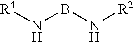

or a salt, hydrate, solvate or N-oxide thereof, wherein:

B is

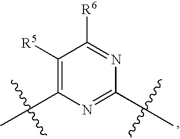

wherein R⁵ and R⁶ together form a saturated or unsaturated alkylene or saturated or unsaturated heteroalkylene chain of 3 to 4 atoms, optionally substituted with one or more R^{a} and/or R^{b}; R² is selected from the group consisting of (C₆-C₂₀) aryl optionally substituted with one or more R⁸, a 5-20 membered heteroaryl optionally substituted with one or more R⁸, a (C₇-C₂₈) arylalkyl optionally substituted with one or more R⁸ and a 6-28 membered heteroarylalkyl optionally substituted with one or more R⁸; R⁴ is a saturated or unsaturated, bridged or unbridged cycloalkyl containing a total of from 3 to 16 annular carbon atoms that is substituted with an R⁷ group, with the proviso that when R⁴ is an unsaturated unbridged cycloalkyl, or a saturated bridged cycloalkyl, this R⁷ substituent is optional, wherein R⁴ is further optionally substituted with one or more R^{f}; R⁷ is selected from the group consisting of —C(O)OR^{d}, —C(O)NR^{d}R^{d}, —C(O)NR^{d}OR^{d}, or —C(O)NR^{d}NR^{d}R^{d}; each R⁸ group is, independently of the others, selected from the group consisting of a water-solubilizing group, R^{a}, R^{b}, C₁-C₈, alkyl optionally substituted with one or more R^{a} and/or R^{b}, —C₃-C₈ cycloalkyl optionally substituted with one or more R^{a} and/or R^{b}, heterocycloalkyl containing 3 to 12 annular atoms, optionally substituted with one or more R^{a} and/or R^{b}, C₁-C₈ alkoxy optionally substituted with one or more R^{a} and/or R^{b}, and —O—(CH₂)ₓ—R^{b}, where x is 1-6; each R^{a} is, independently of the others, selected from the group consisting of hydrogen, C₁-C₈ alkyl, bridged or unbridged C₃-C₁₀ cycloalkyl, bridged or unbridged heterocycloalkyl containing 3 to 12 annular atoms, heteroaryl, (C₆-C₁₄) aryl, and (C₇-C₂₀) arylalkyl, wherein R^{a} is optionally substituted with one or more R^{f}; each R^{b} is, independently of the others, a suitable group selected from the group consisting of ═O, —OR^{a}, (C₁-C₃) haloalkyloxy, ═S, —SR^{a}, ═NR^{a}, ═NOR^{a}, —NR^{c}R^{c}, halogen, —C₁-C₃ haloalkyl, —CN, —NC, —OCN, —SCN, —NO, —NO₂, ═N₂, —N₃, —S(O)R^{a}, —S(O)₂R^{a}, —S(O)₂OR^{a}, —S(O)NR^{c}R^{c}, —S(O)₂NR^{c}R^{c}, —OS(O)R^{a}, —OS(O)₂R^{a}, —OS(O)₂OR^{a}, —OS(O)₂NR^{c}R^{c}, —C(O)R^{a}, —C(O)OR^{a}, —C(O)NR^{c}R^{c}, —C(O)NR^{a}OR^{a}, —C(NH)NR^{c}R^{c}, —C(NR^{a})NR^{c}R^{c}, —C(NOH)R^{a}, —C(NOH)NR^{c}R^{c}, —OC(O)R^{a}, —OC(O)OR^{a}, —OC(O)NR^{c}R^{c}, —OC(NH)NR^{c}R^{c} and —OC(NR^{a})N—R^{c}R^{c}; each R^{c} is, independently of the others, is R^{a} or two R^{c} that are bonded to the same nitrogen atom taken together with the nitrogen atom to which they are both attached form a heterocycloalkyl group containing 5 to 8 annular atoms, which optionally includes from 1 to 3 additional heteroatomic groups selected from the group consisting of —O—, —S—, —N(—(CH₂)_{y}—R^{a})—, —N(—(CH₂)_{y}—C(O)R^{a})—, —N(—(CH₂)_{y}—C(O)OR^{a})—, —N(—(CH₂)_{y}—S(O)₂R^{a})—, —N(v(CH₂)_{y}—S(O)₂OR^{a})— and —N(—(CH₂)_{y}—C(O)NR^{a}R^{a})—, where y is 0-6, wherein the heterocycloalkyl is optionally substituted with one or more R^{f}; each R^{d} is, independently of the others, selected from the group consisting of R^{a}, R^{c} and a chiral auxiliary group; and each R^{f} is independently —C₁-C₈ alkoxy, —C₁-C₈ alkyl, —C₁-C₆ haloalkyl, cyano, nitro, amino, (C₁-C₈ alkyl)amino, di(C₁-C₈ alkyl)amino, phenyl, benzyl, oxo, or halogen, or any two R^{f} bonded to adjacent atoms, taken together with the atoms to which they are each attached, form a fused saturated or unsaturated cycloalkyl or a fused saturated or unsaturated heterocycloalkyl group containing 5 to 8 annular atoms, wherein the formed cycloalkyl and heterocycloalkyl groups are optionally substituted with one or more groups which are each independently selected from halogen, C₁-C₈ alkyl, and phenyl. Compounds of formula I are described and defined in PCT Publication No. WO2007070872A1 and U.S. Patent Publication No. 20070142402.

In one embodiment the bone disorder comprises one or more of osteopenia, osteomalacia, osteoporosis, osteoarthritis, osteomyeloma, osteodystrophy, Paget's disease, osteogenesis imperfecta, bone sclerosis, aplastic bone disorder, humoral hypercalcemic myeloma, multiple myeloma, or bone thinning following metastasis. In one embodiment, the invention provides a method wherein the osteoporosis is post-menopausal, steroid-induced, senile, or thyroxin-use induced.

In one embodiment, the bone disorder is caused by at least one of hypercalcemia, chronic renal disease, kidney dialysis, primary hyperparathyroidism, secondary hyperparathyroidism, inflammatory bowel disease, Crohn's disease, long-term use of corticosteroids, or long-term use of gonadotropin releasing hormone (GnRH) agonists or antagonists.

In one embodiment, the invention provides a method of increasing osteoblast number or osteoblast activity in a mammal, the method comprising administering to the mammal an inhibitor of Axl gene expression in an amount and for a period of time sufficient to increase osteoblast number or osteoblast activity in the mammal. In one embodiment, the invention provides a method wherein the increased osteoblast number or osteoblast activity reduces at least one of: the level of bone deterioration, the loss of bone mass, the loss of bone mineral density, the degeneration of bone quality, and the degeneration of bone microstructural integrity. In one embodiment, the invention provides a method wherein the inhibitor is not BMP2 protein. In one embodiment the invention provides methods wherein the increased osteoblast number or activity results in an increase in expression of an osteoblast marker. In one embodiment the osteoblast marker is osteocalcin, alkaline phosphatase, or collagen type I.

In one embodiment, the inhibitor of Axl gene expression is a compound, a protein, a peptide, an antibody, an aptamer, or a polynucleotide. In one embodiment, the inhibitor of Axl gene expression prevents or reduces Axl gene transcription. In one embodiment, the inhibitor of Axl gene expression prevents or reduces translation of Axl messenger ribonucleic acid (mRNA).

In one embodiment the inhibitor of Axl gene expression is a polynucleotide. In one embodiment the polynucleotide is ribonucleic acid (RNA). In one embodiment the polynucleotide is deoxyribonucleic acid (DNA). In one embodiment the RNA or DNA is antisense. In one embodiment the RNA is double stranded RNA. In one embodiment the double stranded RNA is short interfering RNA (siRNA). In one embodiment the siRNA is about 15 to about 40 nucleotides in length. In one embodiment the siRNA has a nucleotide sequence selected from SEQ ID NO:3, SEQ ID NO:4, SEQ ID NO:5, and SEQ ID NO:6. In one embodiment the siRNA comprises a micro RNA (miRNA) sequence.

In one embodiment, the invention provides a method of increasing osteoblast number or osteoblast activity in a mammal, the method comprising administering to a mammal an inhibitor of Axl protein activity in an amount and for a period of time sufficient to increase osteoblast number or osteoblast activity in the mammal. In one embodiment, the increased osteoblast number or osteoblast activity reduces at least one of: the level of bone deterioration, the loss of bone mass, the loss of bone mineral density, the degeneration of bone quality, and the degeneration of bone microstructural integrity.

In some embodiments, the inhibitor of Axl protein activity is a compound, a protein, a peptide, an antibody, an aptamer, a small molecule immunopharmaceutical (SMIP™), or a polynucleotide. In one embodiment, the inhibitor decreases the tyrosine kinase activity of Axl protein. In one embodiment the inhibitor inhibits interaction between Axl protein and at least one Axl protein ligand. In one embodiment the Axl protein ligand is growth arrest-specific 6 (Gas6) protein; protein S; p855α, or p85β subunits of phosphatidylinositol 3-kinase (PI3K) protein; phospholipase C-γ (PLC-γ) protein, growth factor receptor-bound protein 2 (Grb2); c-Src protein; Ras protein; Akt protein; ERK/MAPK protein; NF-κB protein; GSK3 protein; IL-15 receptor α subunit protein; or mTOR protein. In one embodiment the inhibitor prevents activation of Axl protein by Gas6 protein. In one embodiment the inhibitor binds to the Gas6 major binding site of the Axl protein.

In some embodiments, the inhibitor of Axl protein activity is a soluble Axl protein or a fragment thereof, a mutant Axl protein or a fragment thereof, or an Axl protein ligand or a fragment thereof. In one embodiment the mutant protein is a mutant Axl protein. In one embodiment the mutant Axl protein has a substitution of arginine for lysine at amino acid position 567 of SEQ ID NO:2.

In some embodiments, the inhibitor of Axl protein activity is an antibody. The antibody may be a human antibody or a humanized antibody. In some embodiments the antibody may specifically bind to Axl protein, an Axl protein ligand other than Gas6, the Gas6 major binding site of the Axl protein, or any other site that prevents binding of Gas6 on Axl.

In some embodiments, the invention provides any one or more of the methods as described herein wherein the mammal is a human. In one embodiment, the invention provides methods as described herein wherein the inhibitor of Axl gene expression or the inhibitor of Axl protein activity may be administered systemically. The inhibitor of Axl gene expression or Axl protein activity may be administered repeatedly over a period of time of at least two weeks. In other embodiments, the inhibitor of Axl gene expression or Axl protein activity may be administered locally. The inhibitor may be applied in situ with a matrix.

In some embodiments, the invention provides any one or more of the methods as described herein further comprising administering to the mammal at least one agent selected from the group consisting of a bisphosphonate, a bone morphogenetic protein (BMP), a calcitonin, an estrogen, a selective estrogen receptor inhibitor, a parathyroid hormone, a vitamin, a RANKL inhibitor, a Cathepsin K inhibitor, a sclerostin inhibitor, and strontium ranelate. The BMP may be, e.g., BMP2, BMP4, BMP6, or heterodimers thereof.

In some embodiments, the invention provides a method of treating or preventing a bone disorder in a mammal, the method comprising administering to a mammal an agonist of Axl protein activity or an agonist of Axl gene expression, wherein the bone disorder is associated with increased osteoblast number or increased osteoblast activity. In one embodiment, the disorder may be, e.g., sclerosing bone dysplasia, skeletal bone dysplasia, endosteal hyperostosis, Camurati-Engelmann disease, Van Buchem disease, sclerosteosis, autosomal dominant osteoscleorosis, autosomal dominant osteopetrosis type I, Worth disease, or Fibrodysplasia Ossificans Progressiva.

In some embodiments, the invention provides methods of identifying a compound that modulates bone growth comprising contacting a cell with a test compound, and determining whether Axl gene expression or Axl protein activity in the cell is altered by the compound, wherein alteration of the Axl gene expression or Axl protein activity indicates that the test compound modulates bone growth. In one embodiment, the cell may be, e.g., an osteoblast or an osteoblast precursor. In one embodiment, the test compound inhibits Axl gene expression. In one embodiment, the test compound increases Axl gene expression. In one embodiment, the test compound inhibits Axl protein activity. In one embodiment, the test compound increases Axl protein activity.

In some embodiments, the invention provides methods of identifying a compound that modulates Axl protein kinase activity comprising: providing an Axl polypeptide having kinase activity; providing a substrate which is phosphorylated in the presence of the Axl polypeptide; contacting the polypeptide and substrate with a compound, and determining whether or not the polypeptide modulates Axl kinase activity. In some embodiments, the method of identifying modulators of Axl kinase activity comprises providing an Axl polypeptide having kinase activity; providing a substrate which is phosphorylated in the presence of the Axl polypeptide; mixing the Axl polypeptide and the substrate under conditions which allow phosphorylation of the substrate; contacting the mixture in with a compound; and determining whether or not the compound modulates Axl kinase activity. In some embodiments the Axl polypeptide has the amino acid sequence set forth in SEQ ID NO:13, SEQ ID NO:37, SEQ ID NO:38, SEQ ID NO:39, SEQ ID NO:40, SEQ ID NO:41, or SEQ ID NO:42. In some embodiments the Axl polypeptide has the amino acid sequence set forth in SEQ ID NO:13, SEQ ID NO:37, SEQ ID NO:38, SEQ ID NO:39, SEQ ID NO:40, SEQ ID NO:41, SEQ ID NO:42, or SEQ ID NO:43. In some embodiments, the substrate has the amino acid sequence set forth in SEQ ID NO:16, SEQ ID NO:17, SEQ ID NO:35, or SEQ ID NO:36.

In some embodiments, the invention provides a method of screening or detecting altered bone density in a subject comprising obtaining a test sample from the subject; determining the level of Axl gene expression or the level of Axl protein activity in the test sample; and comparing the level of Axl gene expression or the level of Axl protein activity in the test sample to the level of Axl gene expression or the level of Axl protein activity in a control sample, wherein an altered level of Axl gene expression or Axl protein activity in the test sample relative to the level of Axl gene expression or protein activity in the control sample is indicative of an altered bone density. In some embodiments the level of Axl gene expression or Axl protein activity in the test sample is increased relative to the control sample. In some embodiments, the level of Axl gene expression or Axl protein activity in the test sample is decreased relative to the control sample.

In one embodiment, the invention provides a method of screening or detecting altered bone density in a subject wherein the level of Axl protein activity is determined using a capture reagent that specifically binds Axl protein. In one embodiment, the Axl capture reagent is an antibody. In one embodiment, the antibody is detected using a detectable label. In one embodiment, the detectable label is selected from the group consisting of a radioisotope, a fluorescent compound, a bioluminescent compound and a chemiluminescent compound.

In one embodiment, the invention provides a kit comprising a capture reagent that specifically binds at least one Axl polypeptide, buffer, and reagents for detecting binding of the capture reagent to at least one Axl polypeptide. In one embodiment the capture reagent of the kit comprises a detectable label. In one embodiment, the capture reagent of the kit is an antibody. The kits of the invention can alternatively or additionally comprise nucleic acid probes or primers that are specific for the Axl gene.

In one embodiment, the invention provides a method of screening for altered level of bone mineral density, altered bone mass, altered bone quality, altered bone formation, or altered bone microstructural integrity in a subject comprising determining the presence of at least one mutation in a polynucleotide encoding Axl protein in a test sample from the subject, wherein the presence of said at least one mutation in a polynucleotide encoding Axl protein is indicative of an altered bone density, altered bone mass, altered bone quality, or altered bone formation in the subject.

In one embodiment, the presence or the absence of at least one mutation in a polynucleotide encoding Axl protein is detected by contacting the sample with an oligonucleotide probe that hybridizes specifically with a polynucleotide encoding Axl. In one embodiment, the oligonucleotide probe comprises at least about 15 nucleotides of a polynucleotide encoding an Axl polypeptide. In one embodiment, the polynucleotide is selected from the group consisting of DNA, genomic DNA, complementary DNA (cDNA), RNA, and mRNA. In one embodiment, the polynucleotide encodes a mutant Axl protein. In one embodiment, the mutant Axl protein has a substitution of arginine for lysine at amino acid position 567 of SEQ ID NO:2.

### BRIEF DESCRIPTION OF THE SEQUENCES

The following sequence descriptions and Sequence Listing attached hereto comply with the rules governing nucleotide and/or amino acid sequence disclosures in patent applications as set forth in 37 C.F.R.§.1.821 1.825. The symbols and format used for nucleotide and amino acid sequence data comply with the rules set forth in 37 C.F.R. § 1.822.

SEQ ID NO:1 is the nucleotide sequence of the human Axl gene found in GenBank accession number NM_{—}021913. Nucleotide residues 459 to 3143 encode SEQ ID NO:2.

SEQ ID NO:2 is the amino acid sequence of a full length human Axl found in GenBank accession number NP_{—}068713.

SEQ ID NO:3 is the nucleotide sequence of siRNA Axl 1.

SEQ ID NO:4 is the nucleotide sequence of siRNA Axl 2.

SEQ ID NO:5 is the nucleotide sequence of siRNA Axl 3.

SEQ ID NO:6 is the nucleotide sequence of siRNA Axl 4.

SEQ ID NO:7 is the nucleotide sequence of NSP, a non-specific, scrambled siRNA control.

SEQ ID NO:8 is the nucleotide sequence of siRNA Runx2/Cbfa1.

SEQ ID NOs:9-11 are the nucleotide sequences of the primers used to detect osteocalcin.

SEQ ID NO:12 is the amino acid sequence of a peptide from Axl which contains the protease cleavage site.

SEQ ID NO:13 is the amino acid sequence of a polypeptide having Axl kinase activity.

SEQ ID NOs:14-15 are amino acid sequences of Axl peptides containing autophosphorylation sites.

SEQ ID NOs:16-17 are amino acid sequences of Axl peptides which can be used in screening methods.

SEQ ID NO:18 is the nucleotide sequence of human Axl shRNA construct hAxl 363.

SEQ ID NO:19 is the nucleotide sequence of human Axl shRNA construct hAxl 1107.

SEQ ID NO:20 is the nucleotide sequence of human Axl shRNA construct hAxl 1748.

SEQ ID NO:21 is the nucleotide sequence of human Axl shRNA construct hAxl 1988.

SEQ ID NO:22 is the nucleotide sequence of human Axl shRNA construct hAxl 2448.

SEQ ID NO:22 is the nucleotide sequence of human Axl shRNA construct hAxl 2448.

SEQ ID NO:23 is the nucleotide sequence of mouse Axl shRNA construct mAxl 187.

SEQ ID NO:24 is the nucleotide sequence of mouse Axl shRNA construct mAxl 1079.

SEQ ID NO:25 is the nucleotide sequence of mouse Axl shRNA construct mAxl 1477.

SEQ ID NO:26 is the nucleotide sequence of mouse Axl shRNA construct mAxl 1850.

SEQ ID NO:27 is the nucleotide sequence of mouse Axl shRNA construct mAxl 2269.

SEQ ID NOs: 28-30 are the nucleotide sequences of the siRNAs used to knockdown expression of Axl in the L929 subline to restore sensitivity to TNFα.

SEQ ID NOs 31 and 32 are the nucleotide sequences of the shRNAs used to knockdown Axl and to show that Axl is necessary for ex vivo angiogenesis in a mouse model.

SEQ ID NO:33 is the amino acid sequence of IG1, the major binding site of Gas6 on Axl.

SEQ ID NO:34 is the amino acid sequence of IG2, the minor binding site of Gas6 on Axl.

SEQ ID NO:35 and SEQ ID NO:36 are the amino acid sequences of peptides that may be used in screening methods.

SEQ ID NOs: 37 through SEQ ID NO:42 are the amino acid sequences of polypeptides having kinase activity used in screening methods.

SEQ ID NO:43 is the amino acid sequence of a polypeptide having kinase activity and used in screening methods.

### BRIEF DESCRIPTION OF THE DRAWINGS

FIG. 1 is a graph that shows BMP2 protein downregulates Axl gene expression within 24 hours.

FIG. 2 is a graph that shows that Axl knockdown reduces Axl mRNA levels in Clone 14 cells.

FIG. 3 is a graph that shows that Axl knockdown promotes osteocalcin expression, both in the presence and absence of exogenous BMP2 protein, in Clone 14 cells.

FIG. 4 is a graph that shows Axl knockdown induces alkaline phosphatase activity in Clone 14 cells upon addition of BMP2 protein.

FIG. 5 is a graph that shows that Axl overexpression represses osteocalcin mRNA levels; this effect is enhanced by the addition of BMP2 protein.

FIG. 6 is a graph that shows transient inhibition of Axl/Gas6 binding using an Axl/Fc chimera results in an increase in total bone area and in the number of osteoblasts in an ex vivo murine calvarial organ culture model.

FIG. 7 shows that a “kinase-dead” mutant of Axl does not repress osteocalcin expression in Clone 14 cells.

FIG. 8 shows that 26-week-old Axl male and female knockout mice have increased total, trabecular and cortical bone mass.

### DETAILED DESCRIPTION

The methods of the invention can be used to treat or prevent a bone or cartilage disorder in any mammal in need of such treatment, including, e.g., humans, primates, monkeys, rodents, sheep, rabbits, dogs, guinea pigs, horses, cows, and cats.

The invention provides for an Axl inhibitor to be administered to treat or prevent a bone or cartilage degenerative disorder. The disorders treated or prevented by administration of an Axl inhibitor include, for example, osteopenia, osteomalacia, osteoarthritis, osteoporosis (e.g., post-menopausal, steroid-induced, senile, or thyroxin-use induced), osteomyeloma, osteodystrophy, Paget's disease, osteogenesis imperfecta, humoral hypercalcemic myeloma, multiple myeloma and bone thinning following metastasis. The disorders treated or prevented further include bone degenerative disorders associated with hypercalcemia, chronic renal disease, primary or secondary hyperparathyroidism, inflammatory bowel disease, Krohn's disease, long-term use of corticosteroids or gonadotropin releasing hormone (GnRH) agonists or antagonists, and nutritional deficiencies.

The invention provides methods of administering to a mammal an inhibitor of Axl in an amount effective to treat or prevent a bone degenerative disorder; slow bone deterioration; restore lost bone; stimulate new bone formation; and/or maintain bone (bone mass and/or bone quality).

The invention provides methods to treat microdefects in trabecular and cortical bone. Bone quality can be determined, for example, by assessing microstructural integrity of the bone.

The invention provides methods to treat or prevent a bone degenerative disorder in a post-menopausal woman. The invention provides methods to treat or prevent a bone degenerative disorder in a man. The invention provides methods to treat or prevent a bone degenerative disorder in an individual with steroid-induced osteoporosis. The invention provides methods to treat or prevent senile osteoporosis in an individual. The invention provides methods to treat or prevent thyroxin-use or glucocorticoid-use induced osteoporosis in an individual.

The invention provides for an Axl agonist to be administered to treat or prevent a bone disorder characterized by excessive bone growth or skeletal overgrowth. For example, bone disorders characterized by excessive bone growth or skeletal overgrowth include, but are not limited to, e.g., sclerosing bone dysplasia, also termed “sclerosteosis”; skeletal bone dysplasias, such as osteosclerosis, osteopetrosis, and endosteal hyperostosis; Camurati-Engelmann disease; Van Buchem disease and sclerosteosis; autosomal dominant osteosclerosis; autosomal dominant osteopetrosis type I; Worth disease; and fibrodysplasia ossificans progressiva (FOP). See, e.g., Wesenbeck et al., Am. J. Human Genet. 72: 763-771 (2003), and references cited therein. Other forms of excessive bone growth include the pathological growth of bone following hip replacement surgery, trauma, burns, or spinal cord injury, as well as excessive bone growth associated with metastatic prostate cancer or osteosarcoma.

The invention provides methods for enhancing a BMP2-mediated response in a mammal, by co-administering BMP2 and an inhibitor of Axl to the mammal. BMP2 is a potent osteogenic agent that is useful for the treatment of patients who exhibit bone and cartilage defects (see for example U.S. Pat. Nos. 5,166,058 and 6,150,328). Thus, the invention provides methods of co-administering BMP2 and an Axl inhibitor to treat or prevent any of the bone degenerative disorders described above including, e.g., osteopenia, osteomalacia, osteoarthritis, osteoporosis, osteomyeloma, osteodystrophy, Paget's disease, osteogenesis imperfecta, humoral hypercalcemic myeloma, multiple myeloma and bone thinning following metastasis, as well as bone degenerative disorders associated with hypercalcemia, chronic renal disease, primary or secondary hyperparathyroidism, inflammatory bowel disease, Krohn's disease, and long-term use of corticosteroid. The invention also provides methods of co-administering BMP2 and an Axl inhibitor to treat or prevent any additional conditions treated or prevented by BMP2. The methods include BMP2 treatment of bone fracture and augmentation of spinal fusion.

Outcome(s) related to bone deterioration may be evaluated by a specific effect of the Axl modulator with respect to loss of trabecular bone (trabecular plate perforation); loss of (metaphyseal) cortical bone; loss of cancellous bone; decrease in bone mineral density; reduced bone mineral quality; reduced bone remodeling; increased level of serum alkaline phosphatase and acid phosphatase; osteocalcin expression; bone fragility (increased rate of fractures); and decreased fracture healing. Methods for evaluating these outcomes are provided in detail below. Bone deterioration and/or bone mass augmentation can be assessed in vivo using densitometric imaging, including radiography, dual energy X-ray absorptiometry (DXA) or quantitative computed tomography (QCT). Bone quality can be measured ex vivo using high-resolution densitometric imaging methods that provide detailed information on bone microstructure such as micro-computed tomography (microCT), or biomechanical testing of bone to determine fracture resistance.

Additional applications of the present invention include the use of Axl modulators for coating, or incorporating into, osteoimplants, matrices, and depot systems so as to promote osteointegration. Examples of such implants include dental implants, joint replacements implants and bone graft substitutes.

The formulations may also include an appropriate matrix, for instance, for delivery and/or support of the composition and/or providing a surface for bone and/or cartilage formation. The matrix may provide slow release of the inhibitor of Axl gene expression or the inhibitor of Axl protein activity or other cartilage/bone protein or other factors of the formulation and/or the appropriate environment for presentation of the formulation of the invention. For bone and/or cartilage formation, the composition would include a matrix capable of delivering the compositions to the site of intended use. Such matrices may be formed of materials presently in use for other implanted medical applications.

The choice of matrix material is based on one or more of biocompatibility, biodegradability, mechanical properties, cosmetic appearance and interface properties. Potential matrices for the compositions may be biodegradable and chemically defined calcium sulfate, tricalciumphosphate, hydroxyapatite, polylactic acid and polyanhydrides as well as coral. Further matrices are comprised of pure proteins or extracellular matrix components. Other potential matrices are nonbiodegradable and chemically defined, such as sintered hydroxyapatite, bioglass, aluminates, or other ceramics. Matrices may be comprised of combinations of any of the above-mentioned types of material, such as polylactic acid and hydroxyapatite or collagen and tricalciumphosphate. The bioceramics may be altered in composition, such as in calcium-aluminate-phosphate and processing to alter pore size, particle size, particle shape, and biodegradability.

The invention comprises assays for evaluating the efficacy of an Axl modulator for treatment of a bone degenerative disorder. Such an assay comprises administering the modulator repeatedly to a mammal (e.g., an OVX rat) for a period of at least 2, 4, 6, or 8 weeks; and determining the effect of the modulator on bone, wherein a slowing of bone deterioration (e.g., bone mass and/or bone quality) or increase in bone formation attributable to the modulator indicates that the modulator is effective for treatment or prevention of a bone degenerative disorder, and wherein decreased bone density attributable to the modulator indicates that the modulator is effective for treatment of a sclerosing bone dysplasia or disorders of inappropriately elevated bone mass.

Assays to Measure Effects of Axl Modulators on Bone

The effect of an Axl modulator on different aspects of bone structure and bone formation may be measured by methods including, but not limited to, skeletal phenotyping assays, which assess bone mass, bone quality, bone density, bone formation, and bone deterioration; animal models of bone disorders; and in vitro tests, including assays for newly formed bone (e.g. calcein-labelled), osteocalcin gene expression, as well as alkaline phosphatase activity.

As used herein, “skeletal phenotyping” refers to the characterization of bone(s) by using one or more assays that assess bone mass, including bone mineral density and/or bone quality. Such assays can measure loss of trabecular bone (trabecular plate perforation), loss of (metaphyseal) cortical bone, loss of cancellous bone, decrease in bone mineral density, reduced bone mineral quality, reduced bone remodeling, increased level of serum alkaline phosphatase and acid phosphatase, bone fragility (increased rate of fractures), and decreased fracture healing. These assays can also measure increased bone mass due to anabolic bone formation, including increases in trabecular number or trabecular thickness, increase in cortical bone, increase in bone mineral density, increased level of serum osteocalcin and alkaline phosphatase, improved mechanical integrity and augmented or accelerated fracture healing.

The invention provides methods for measuring the effect of an Axl modulator on bone mass and quality, including bone mineral density (BMD). Methods for evaluating bone mass and quality are known in the art and include, e.g., X-ray diffraction, DXA, pDXA, DEQCT, CT, PQCT, chemical analysis, density fractionation, histophotometry, histomorphometry, and histochemical analysis as described, for example, in Lane et al., J. Bone Min. Res. 18:2105-2115 (2003).

One method for measuring the effect of an Axl modulator on bone mineral density is dual energy x-ray absorptiometry (DXA) and/or peripheral DXA (pDXA). Though it can be used for measurements of any skeletal site, clinical determinations are usually made of the lumbar spine and hip. Portable DXA machines have been developed that measure the heel (calcaneus), forearm (radius and ulna), or finger (phalanges). DXA can also be used to measure body composition. In the DXA technique, two x-ray energies are used to estimate the area of mineralized tissue, and the mineral content is divided by the area, which partially corrects for body size. However, this correction is only partial, because DXA is a two-dimensional scanning technique and cannot estimate the depths or posteroanterior length of the bone. Thus, small people tend to have lower-than-average bone mineral density (BMD). Newer DXA techniques that more accurately measure BMD are currently under development. Bone spurs, which are frequent in osteoarthritis, tend to falsely increase bone density of the spine. Because DXA instrumentation is provided by several different manufacturers, the output varies in absolute terms. Consequently, it has become standard practice to relate the results to “normal” values using T-scores, which compare individual results to those in a young population that is matched for race and gender, but not age. Alternatively, Z-scores compare individual results to those of an age-matched population that is also matched for race and gender. Thus, a 60-year-old woman with a Z-score of −1 (1 standard deviation (SD) below mean for age) could have a T-score of −2.5 (2.5 SD below mean for a young control group). pDXA is also useful for measuring BMD in laboratory animals such as rats and mice.

Methods for measuring the effect of an Axl modulator on bone microstructure using micro-computed tomography (μCT or MicroCT) are known in the art. MicroCT is a method that produces 3600 radioscopic image data on an object by turning the apparatus while irradiating the object with X-rays. The data is then used to generate a fully 3-dimensional image dataset from which trabecular and cortical bone volume can be measured. Because of its superior spatial resolution, μCT detects changes in the trabecular structure of bone that are not observable by DXA or pDXA. This assay can provide more insight into mechanical properties of bone because it depends, not only on BMD as quantified by DXA and pDXA, but also on the spatial arrangement of trabeculae in trabecular bone, which may be measured by μCT.

Methods for measuring the effect of an Axl modulator on BMD using peripheral quantitative computed tomography (PQCT) are available. In the PQCT method, volumetric BMD (vBMD, mg/cm³) of the proximal tibiae (but not limited to) for example can be evaluated in anesthetized rats using an XCT-960M instrument (XCT Research, Stratec Medizintechnik, Pforzheim, Germany). A 1 mm-thick PQCT slice obtained 3.4 mm distal from the proximal end of the tibia is used to compute total and trabecular density for the proximal tibial metaphysis. The tomographic slice has an in-plane voxel (three dimensional pixel) size of 0.140 mm. After acquisition, the image is displayed and the region of interest including tibia but excluding fibula is outlined. The soft tissue is automatically removed using an iterative algorithm, and the density of the entire bone (total density, mg/cm³) in the slice is determined. For trabecular density determination, the outer 55% of the bone slice is then peeled away in a concentric spiral and the value of the trabecular density is reported in mg/cm³.

The effect of an Axl modulator on bone formation may be measured, e.g., using calcein labeling. For example, mice can be injected with calcein (e.g. 15 mg/kg, 0.1 ml/mouse, s.c.) at nine and two days prior to tissue collection. Bone tissues can be collected from, either, femora, tibiae, as well as spine. Histological characterization of bone samples measures the distance between calcein-labeled mineralized bone layers and is used to evaluate bone formation.

The invention provides methods for evaluating the effect of an Axl modulator in one or more animal models of bone disorders, including bone degenerative disorders, and/or in humans. Osteopenia may be induced, for example, by immobilization, low calcium diet, high phosphorus diet, long-term use of corticosteroid, or gonadotropin releasing hormone (GnRH) agonist or antagonist, cessation of ovary function, or aging. For example, ovariectomy (OVX)-induced osteopenia is a well-established animal model of human post-menopausal osteoporosis. Another well-validated model involves administration of corticosteroids. Such animal models include: cynomolgus monkeys, dogs, rats, mice, rabbits, ferrets, guinea pigs, minipigs, and sheep. For a review of various animal models of osteoporosis, see, e.g., Turner, Eur. Cell. Mater. 1:66-81 (2001).

Appropriate in vivo and in vitro tests for the evaluation of the effect on osteoblasts in culture such as the effect on collagen synthesis and osteocalcin expression or the effect on the level of alkaline phosphatase and cAMP induction are described in, for example, U.S. Pat. No. 6,333,312.

Cells useful in developing the invention include osteoblasts and osteoblast precursors. Specifically, these cells may include mesenchymal stem cells, osteoprogenitor cells derived from bone marrow, and osteoprogenitor cells circulating in blood. Useful in practicing the methods of the invention are skeletal bone cells including osteoprogenitor cells, bone lining cells, osteoblasts, osteocytes. Cell types that may also be used include embryonic fibroblasts, myoblastic precursors or adipocyte lineage (which would include pre-adipocyte). Immortalized or transformed cells may be used in vitro to evaluate the activity of a compound or therapeutic agent as a modulator of Axl gene expression or protein activity before testing the compound or therapeutic agent vivo animal models.

Bone-specific alkaline phosphatase is a membrane-bound enzyme located on the outer cell surface. It is an osteoblast lineage-specific marker of osteoblast activity associated with early phases of osteogenesis. Alkaline phosphatase activity may be examined qualitatively by histochemical staining with a mixture of naphthol AS-MX phosphate and fast blue BB salt. Results of the staining may be recorded using bright-field microscopy, noting blue-stained cells or colonies indicating cells of the osteoblast lineage. Alkaline phosphatase activity may be determined quantitatively by a colorimetric enzymatic assay. Activity is assayed in cell lysates using p-nitrophenyl phosphate as a substrate, and measured by taking absorbance readings at 405 nm. Absorbance data is compared to appropriate controls and normalized to account for variation in protein yield between sample isolates. Alkaline phosphatase levels are determined relative to a standard curve that is generated using known amounts of alkaline phosphatase enzyme. Values are then normalized to total cellular protein and compared between samples. Variations on these assays, as well as additional methods of measuring alkaline phosphatase activity, are well within the knowledge of a practitioner having ordinary skill in the art (see, e.g., Cheng et al., J. Bone Joint Surg. 85:1544-1552 (2003))

Osteocalcin is the most abundant non-collagenous protein in bone and is produced specifically by mature osteoblasts. Osteocalcin is used as a marker of osteoblast-specific activity during the later phases of differentiation. Thus, an Axl gene expression modulator or an Axl protein activity modulator may modulate the osteocalcin levels. Osteocalcin gene expression may be measured by Northern blotting, as described in detail below. Osteocalcin gene expression may be measured by real-time RT-PCR or may be assayed using a widely available radioimmunoassay kit (Biomedical Technologies, Inc, Stoughton, Mass.). Other methods of detecting and quantifying osteocalcin gene expression are well known to persons of ordinary skill in the art (see, e.g., Thies et al., Endocrinol. 130:1318-1324 (1992)).

Matrix mineralization is associated with terminally differentiated osteoblasts. Before assaying mineralization, mesenchymal stem cells and or osteoprogenitor cells are first grown in culture and optionally treated with an osteogenic agent, for example, a BMP. Mineralization of cells may be assessed by calcium isotope accumulation, by histochemical staining, or by other methods well known to persons of ordinary skill in the art. The cells can be incubated for 48 hours in medium containing 0.5 μCi/ml of ⁴⁵CaCl₂ added at various time points after seeding. Cell monolayers are then washed twice with PBS using 1 ml per wash. Next, cells are harvested, digested in 0.1 N NaOH and aliquots are counted by liquid scintillation counting using a Beckman 5500 scintillation counter. Calcified nodules in actively mineralizing cultures are visualized by staining cell monolayers with Alizarin-Red-S. Cell cultures are washed twice with PBS, fixed for 10 minutes in 50% ethanol, rehydrated with 1 ml of distilled water for 5 minutes and then stained for 1-3 minutes with 200 μL of a 1% (w/v) aqueous solution of Alizarin Red S. The monolayers are then washed with distilled water, and the presence of calcified nodules determined by light microscopy. The presence of red-stained colonies of cells by under light microscopy indicates mineralization.

### Axl Modulators

The methods of the invention include administration of modulators of Axl gene expression or Axl protein activity to treat or prevent cartilage and bone disorders. These modulators may increase or decrease Axl gene expression or Axl protein activity.

### Axl

The Axl receptor tyrosine kinase was identified as a protein encoded by a transforming gene from primary human leukemia cells (O'Bryan et al., Mol. Cell. Biol. 11:5016-5031 (1991); Janssen et al., Oncogene 6:2113-2120 (1991); Genbank Accession No. M76125). The Axl receptor tyrosine kinase is synthesized as a 887 amino acid polypeptide, including an 18 amino acid signal peptide (Genbank Accession No. P30530). Full length, transmembrane bound human Axl receptor protein is 140 kDa. In addition, Axl protein can be post-translationally processed by cleavage in a 14 amino acid region immediately N-terminal to the transmembrane domain, generating an 80 kDa soluble extracellular domain (ECD), also called soluble Axl (sAxl), and a 55 kDa membrane-bound kinase domain (O'Bryan et al., J. Biol. Chem. 270:551-557 (1995)). The structural and functional aspects of Axl, as well as its ligands, are well known in the art (see, for example, Heiring et al., J. Biol. Chem. 279:6952-6958 (2004); Budagian et al., Mol. Cell. Biol. 25:9324-9339 (2005)).

The term “Axl gene”, as used herein, refers to any of the genes encoding one or more isoforms of Axl protein, including fragments having Axl protein activity. The nucleotide sequence in Genbank Accession No. NM_{—}021913 is a 5014 bp mRNA encoding the full length human Axl protein isoform 1. The polynucleotide sequence of a 4987 bp mRNA encoding Axl protein isoform 2 is found in Genbank Accession No. NM_{—}001699.

The terms “Axl protein”, or “Axl polypeptide”, as used herein, refer to any one or more isoforms, including proteolytic cleavage products and fragments that have functional activity, of the Axl protein. The 894 amino acid sequence of the full length Axl protein, also referred to as isoform 1, is found in Genbank Accession No. NP_{—}068713. Axl isoform 2 is a 885 amino acid protein (Genbank Accession No. NP_{—}001690) which lacks an internal nine amino acids encoded by exon 10, which are immediately N-terminal to the protease cleavage site (see; O'Bryan et al., J. Biol. Chem. 270:551-557 (1995)). In addition to the two Axl protein isoforms, both human and mouse Axl proteins undergo proteolytic processing near the transmembrane domain to yield a soluble form of the protein, as described above (O'Bryan et al., J. Biol. Chem. 270:551-557 (1995); Costa et al., J. Cell. Physiol. 168:737-744 (1996); Budagian et al., Mol. Cell. Biol. 25:9324-9339 (2005)). Thus, as used herein, the term “Axl protein” refers to the full length transmembrane bound Axl receptor, as well as the forms resulting from post-translational cleavage. As used herein, the term “sAxl protein,” also known as soluble Axl, refers to the extracellular domain cleavage product; the term “membrane bound kinase domain” refers to the membrane bound cleavage product. The term “Axl-ECD” as used herein refers to the Axl extracellular domain.

Axl protein, including its isoforms, may be present as a monomer, homodimer, or in a heterodimer, for example, with an Axl ligand such as Gas6. Dimers include homodimers of the full length, membrane bound protein as well as sAxl-sAxl homodimers, Axl-sAxl heterodimers, Axl-Gas6 heterodimers, and sAxl-Gas6 heterodimers. Depending on conditions, the mature Axl protein may establish equilibrium between any or all of these different forms. “Axl protein” or “Axl polypeptide” also refers to biologically active forms of Axl protein, including any fragments and variants that maintain at least some biological activities associated with Axl protein. For example, an Axl protein can include a peptide fragment having the minimal amino acid sequence required to provide kinase activity. The present invention relates to Axl protein from all vertebrate species including, e.g., human, bovine, chicken, mouse, rat, porcine, ovine, turkey, baboon, and fish.

The term “Axl ligand,” unless otherwise indicated, refers to any ligand that binds at least one Axl protein isoform. One Axl ligand is growth-arrest-specific gene 6 (Gas6) protein (Stitt et al., Cell 80:661-670 (1995); Varnum et al., Nature 373:623-626 (1995); U.S. Pat. No. 5,538,861). Gas6 is a vitamin K-dependent protein with 44% sequence identity to human protein S. Gas6 has a gamma-carboxyglutamic acid rich region, four epidermal growth factor-like repeats, and a carboxy-terminal putative steroid binding domain (Manfioletti et al., Mol. and Cell. Biol. 13:4976-4985 (1993)). In addition to Axl protein, Gas6 binds Tyro-3 and Mer, the other members of the Mer family of receptors (Nagata et al., J. Biol. Chem. 271:30022-30027 (1996); Crossier et al., Pathology 29:131-135 (1997)). Based on the crystal structure of family member Tyro3, sequence homology, and mapping of conserved residues, Gas6 likely binds to the first two immunoglobulin domains of Axl, particularly to the conserved surface patch on domain 2 close to the interdomain interface (Heiring et al., J. Biol. Chem. 279:6952-6958 (2004)).

### Assays for Axl Activity

The terms “Axl protein activity” or “active Axl protein” refer to one or more biological activities associated with active Axl protein. Axl protein activity includes, e.g., tyrosine kinase activity, binding to GAS6, activating or binding other Axl molecules themselves, binding other downstream targets. As used herein, the term “tyrosine kinase activity” (as in the tyrosine kinase activity of Axl) refers to the transfer of a phosphate group from ATP to a tyrosine residue in a protein substrate. As described herein, Axl also has musculoskeletal activities associated with its effects on bone growth.

Assays for measuring Axl protein activity, including tyrosine kinase activity, in vivo and in vitro are known in the art. Examples of some of the more frequently used bioassays include but are not limited to the following:

### Screening for Axl Receptor Tyrosine Kinase Activity

There are numerous kinase enzyme assays platforms known in the art that can be used to identify kinase activators or inhibitors. Examples of kinase enzyme assays for Axl kinase activity would include utilizing time-resolved fluorescence energy transfer (TR-FRET) methodology including Lanthascreen™ (Invitrogen, Carlsbad, Calif.), Lance, and AlphaScreen® (PerkinElmer, Inc., Wellesley Mass.) assays. In an example, using a 96 well or 384 well plate, the substrate peptide which could include either one of the Axl autophosphorylation peptides (5-FAM-DCLDGLYALMSRC (SEQ ID NO:16) or 5-FAM-KKIYNGDYYRQG (SEQ ID NO:17)) or a non-specific peptide (poly Glu:Tyr (4:1) Invitrogen Catalog No. PV3610) is added to assay buffer containing 40 mM MOPS, pH7.0, and 7.2 mM MgCl₂. Then, ATP and Axl (a fragment comprising the kinase domain, in 20 mM MOPS, pH7.0, 0.01% Brij-35, 5% glycerol, 0.1% beta mercapto-ethanol) are added to final concentrations of 50 nM peptide, 50 μM ATP and 5 nM Axl. After incubation at room temperature for 1 hr the reaction is stopped with the addition of 60 mM EDTA. The anti-phosphotyrosine antibody (Invitrogen, Catalog No. PV3552) is added to the reaction mixture at a final concentration of 2.5 nM. After a further 30 minute incubation at room temperature the plate is read following excitation at 340 nM (the excitation wavelength of the terbium donor). The energy transfer to fluorescein without interference from terbium is achieved by measuring the emission in the silent region between the two terbium peaks using a 520 nm filter. This emission is then typically referenced to the emission of the first terbium peak using a 495 nm filter. In this assay, compounds will be identified that either dose dependently reduce the formation of phosphorylated product as indicated by a decrease in the FRET value (antagonist) or increase the FRET value (agonist). In another example, using a 96 or 384-well plate, a LANCE TR-FRET assay is conducted as follows: the substrate peptide AGAGGPQDIYDVPPVR (set forth in SEQ ID NO:36) bound to biotin is added to assay buffer containing 50 mM HEPES pH 7.1, 10 mM MgCl₂, 1 μM BSA, 0.023 mM Brij35 and 11% glycerol. Then, ATP and Axl enzyme (kinase domain) are added to final concentrations of 500 μM ATP, 10 nM Axl and 250 nM substrate peptide. The reaction is then incubated for 45 minutes at 23° C., after which the reaction is stopped by the addition of EDTA in assay buffer to a final concentration of 12 mM. Then, 2 nM Europium-labeled PT66 anti-phosphotyrosine antibody (Invitrogen) and 50 nM Streptavidin-Allophycocyanin (APC) are added and the mixture incubated at room temperature for 90-100 minutes, after which the amount of phosphorylated substrate is determined using a suitable plate reader (e.g. Envision, View Lux, Victor). The Streptavidin-APC binds to the biotin moiety of the substrate. When Europium labeled antibody binds to the phosphorylated tyrosine of the substrate the Europium is now in close proximity to the APC. Under these circumstances, excitation of the complex at 340 nm excites the Europium, which then emits light with a peak wavelength of 615 nm. This in turn excites APC that is in sufficiently close proximity (i.e. because it is bound to the same substrate molecule) to emit light at a wavelength of 665 nm. Hence, light emission at 665 nm provides a measure of the amount of phosphorylated substrate. This value is normalized (by simple ratio) to the 615 nm signal from the unbound antibody. Inhibitory (antagonist) compounds cause a reduction in the amount of the 665/615 nm signal, compared to that generated in the absence of compound, which can be expressed either as a percent inhibition or, in dose-response format, as an IC50; whereas stimulatory compounds (agonists) cause an increase in the 665/615 nm signal, which can be expressed as a percent stimulation or an EC50.

An indirect TR-FRET based approach would be using a Transcreener Kinase assay developed by BellBrook labs which measures the level of ADP generated in the kinase reaction. In this assay, an antibody developed to detect ADP is labeled with terbium. Using a fluorescein labeled ADP tracer (when bound to the ADP antibody-terbium, results in high FRET signal), as the substrate is phosphorylated the levels of unlabeled ADP increase displacing the ADP-tracer from the antibody resulting in a decrease in FRET signal. Therefore, in this assay a kinase inhibitor is expected to increase the FRET signal dose dependently, whereas an agonist would result in a decrease in FRET signal. Alternatively, kinase activity could be measured by consumption of P³³-labeled ATP. In this method Axl or a fragment containing the Axl kinase domain is combined with MgCl₂, P³³-labeled ATP, and substrate bound to a 0.2 μm filter. Transfer of radiolabeled phosphate by Axl onto the filter-bound substrate generates detectable filter-bound radioactivity which reflects the level of substrate phosphorylation.

Modulation of Axl activity can also be assayed within a cell. Such assays include, among others, measurement of Axl autophosphorylation in a phospho-blot; measurement of phosphorylation of downstream targets of Axl; and measurement of cell growth in cells engineered to be dependent upon Axl kinase activity. For example, Axl autophosphorylation can be detected following GAS6 stimulation of Axl-containing cells such as the human glioblastoma cell line A172, using a technique such as ELISA (kit DYC2228, R & D Systems, Minneapolis, Minn.) or phospho-blot in which the phosphorylated Axl is detected, following immunoprecipitation with an anti-Axl antibody, by Western blot using anti phosphotyrosine antibody. Axl inhibitory compounds (antagonists) result in reduced levels of phosphorylated Axl, whereas Axl stimulators (agonists) result in higher levels. Also, Axl kinase activity can be assayed by measuring the effects on protein targets that are affected by Axl activity, directly or indirectly. One such example is Akt, which is downstream of Axl in the Axl/Gas6/PI3Kinase/Akt survival pathway (Weinger et al, J. Neurochem. April 14 epub (2008)). Akt is phosphorylated following GAS6 stimulation of Axl (Shankar et al. J. Neurosci. 26:5638-5648 (2006)). Akt phosphorylation in cells can be detected either by phospho-blot or alpha screen (SureFire™ assay, Perkin Elmer, Waltham, Mass.) using antibodies to the phospho-Thr308 Akt or phospho-Ser473 Akt. Axl inhibitory compounds (antagonists) result in reduced levels of phosphorylated Akt, whereas Axl stimulators (agonists) result in higher levels of phosphorylated Akt. Also, cells can be engineered to be dependent upon Axl kinase activity for their growth. For example, 32D cells, which are usually dependent upon IL-3 for their growth, can be engineered to be dependent upon Axl kinase activity instead of IL-3. This is achieved by transfecting 32D cells with a vector comprising a transforming v-Src N-terminal sequence (including the unique, SH2 and SH3 domains of v-src) spliced to the kinase domain of Axl, and the GFP marker protein. The cells are then grown in the absence of IL-3. GFP positive cells that continue to grow in the absence of IL-3 are dependent upon Axl kinase activity for their growth. Growth can easily be assayed using standard methods e.g. Cell-Titer Glo (Promega, Madison, Wis.) that measures cellular ATP. Axl inhibitory compounds (antagonists) result in reduced levels of 32D cell growth and hence ATP, whereas Axl stimulators (agonists) result in increased levels of growth and hence ATP. Cell based assays can also be utilized to measure Axl activity by taking advantage of cellular changes elicited by the molecular actions of Axl. For example, Budagian et al. (EMBO J. 24:4260-4270, (2005)) has demonstrated that Axl protein protects murine L292 cells from tumor necrosis factor α (TNFα)-induced cell death through its interaction with interleukin-15 receptor α subunit (IL-15Rα). Therefore, a cell-based assay can be developed to identify compounds that modulate Axl kinase activity by measuring L292 cell death. Specifically, L292 cells stably overexpressing Axl would be treated with TNFα in the presence of, for example, a small molecule antagonist of Axl kinase. This would result in a dose-dependent decrease in cell number (increase in cell death). Cell number and/or cell death can be measured with commercially available assays (such as those available from Promega) that measure cellular ATP (indirect measure of cell number-CellTiter Glo® assay) or by cellular LDH release indicating cell death (CytoTox-One™ assay). Similar cellular functional assays can be developed taking advantage of Gas6 induced Axl mediated chemotaxis of vascular smooth muscle cells (Fridell et al. J. Biol. Chem. 273:7123-7126 (1998)) or Gas6 induced Axl mediated aggregation of 32D myeloid cells (McCloskey et al. J. Cell. Biol. 272:23285-23291 (1997)).

Assays to Identify Molecules that Modulate Axl:Gas6 Interaction

Assays that can be used to identify molecules that interact with Axl or can modulate Gas6 binding to Axl include, but are not limited to, Enzyme-Linked ImmunoSorbent (ELISA) Assays, co-immunoprecipitation (Co-IP) assays and Biacore® assays. One skilled in the art is familiar with these assays. Specifically, an ELISA-based method involves immobilizing either Axl protein (or a fragment thereof) or Gas6 protein to a solid support such as nylon, nitrocellulose membrane, a silicon chip, a glass slide, beads or specifically designed assay plates. With one protein bound (e.g. Axl) the ligand (e.g. Gas6) is added in the presence or absence of pharmaceutical molecule (small molecule, antibody, peptide, etc.) and incubated for a length of time to allow interaction. The plate is then washed to remove the unbound Gas6 protein and the remaining protein is detected either directly if Gas6 is labeled (e.g. fluorescently, radioactively, or conjugate with enzyme like alkaline phosphatase or biotin) or indirectly with Gas6 specific antibodies. The interaction that is either enhanced or inhibited by the pharmaceutical molecule is quantitated typically by a colorimetric readout or by fluorimetric endpoint. A similar assay described by Budagian et al. (EMBO J. 24: 4260-4270 (2005)) has been used to demonstrate Axl interaction with IL-15Rα. A solution phase assay (Co-immunoprecipitation) can also be performed using either biotinylated protein, antibodies to the epitope tagged protein (V5, flag, GST, Fc, etc), or protein-specific antibodies whereby the protein/antibody complex is captured, using for example, protein A or protein G (which binds antibody), or in the case of a biotinylated protein, avidin conjugated sepharose beads would be utilized. Any interacting protein is subsequently pulled down in the complex and the interacting protein is identified by polyacrylamide gel electrophoresis and subsequent western blotting. A similar protocol has been described for Axl by Nagata et al. J. Biol. Chem. 271:30022-30027, 1996 and Goruppi et al., Mol. Cell. Biol. 17:4442-4453, 1997.

Identification of therapeutic compounds that can modulate the interaction of Axl protein (or fragments thereof) with Gas6 can be achieved, e.g., by plasmon resonance spectroscopy observation using an instrument such as those made by Biacore® (Uppsala, Sweden). In this method a protein (e.g. Axl) is bound to a sensor chip and a test compound added. The second protein (e.g. Gas6) is added under conditions which permit the two proteins to interact. The output signal of the instrument provides an indication of any effect exerted by the test compound on the interaction of the two proteins (e.g. Axl and Gas6). A similar protocol has been described for Axl by Nagata et al. J. Biol. Chem. 271:30022-30027, 1996.

Cell based binding assays are also commonly used and known in the art. Specifically an Axl binding assay could be developed by transiently overexpressing Axl protein or by developing a stable cell line using, for example, murine L929 cells which express endogenous Axl but minimally express Tyro3 and Mer receptor tyrosine kinases. Approximately 40,000 cells per reaction are washed twice with the assay buffer (DMEM high glucose, 25 mM HEPES, 1 μg/ml heparin, 1% bovine serum albumin (BSA)). An appropriate volume of unlabelled Gas6 protein is added at 100-fold excess of [125I] Gas6 in assay buffer (NSB), and a pharmaceutical molecule, e.g., a small molecule, a peptide, or an antibody, is then added in a treatment or vehicle buffer to the appropriate wells. An appropriate volume of the [125I] Gas6 is then added to all the wells and the cells are incubated for 3 hours at room temperature, 22° C. The cells are washed 2 times with the assay buffer by inversion and add 100 μl 0.5% SDS in PBS is added to lyse the cells. The lysate is then collected and the radiation measured in a gamma radiation counter. Specific binding is calculated by subtracting binding obtained in the presence of unlabeled Gas6 from the total binding value.

### Axl Protein Modulators

The Axl protein modulators for use in the methods of the invention modulate a biological activity of Axl and have a desired effect on bone. The modulator may be an inhibitor of Axl protein and increases bone density, bone mass, bone quality, and/or bone formation. The modulator may be an agonist of Axl protein and decreases bone density, bone mass, bone quality, and/or bone formation. The effect of a modulator on the expression of Axl protein can be determined by any one of the methods known in the art to measure gene expression, some of which are described below. The effect of a modulator on the tyrosine kinase activity of Axl can be determined using any of the assays described above. The effect of a modulator on a musculoskeletal activity of Axl, such as its effect on bone density, bone mass, bone quality, and/or bone formation, can be determined using any of the assays described above.

The term “Axl inhibitor,” “antagonist,” “neutralizing,” and “downregulating” refer to a compound (or its property, as appropriate) which acts as an inhibitor of Axl relative to its activity in the absence of the same inhibitor. The term “direct Axl inhibitor” generally refers to any compound that directly downregulates the biological activity of Axl by interacting with an Axl gene, an Axl transcript, an Axl protein, or an Axl ligand. As used herein, the term “inhibits a biological activity of Axl” refers to a condition (e.g., the addition of an inhibitor of the present invention) that reduces a biological activity of Axl by at least about 15 percent, preferably by at least 50 percent, more preferably by at least 90 percent, and most preferably at least 99 percent. The biological activity can be measured using any suitable method including, but not limited to, the methods described above.

The term “Axl agonist,” “increasing,” and “upregulating” refer to a compound (or its property, as appropriate) that acts as an agonist of a biological activity of Axl protein. As used herein, the term “increases the tyrosine kinase activity of Axl” refers to a condition (e.g., the addition of an agonist of the present invention) that increases the tyrosine kinase activity of Axl protein by at least about 15 percent, preferably by at least 50 percent, more preferably by at least 90 percent, and most preferably at least 99 percent.

The term “kinase-dead” Axl refers to an Axl protein where the conserved lysine in the ATP binding site has been mutated by substitution of arginine for lysine at amino acid position 567 inactivating the enzymatic activity of the kinase.

An Axl protein inhibitor may, for example, inhibit Axl by at least any of the following: (1) inhibiting the kinase activity of Axl; (2) decreasing Axl expression levels; (3) affecting stability of the transmembrane Axl receptor or soluble Axl; (4) affecting cleavage of full length transmembrane-bound Axl to soluble Axl; (5) interfering with the binding of an Axl protein ligand, such as Gas6, to Axl; (6) interfering with dimerization of Axl; or (7) interfering with intracellular signaling of the Axl receptor.

The Axl protein inhibitor may be an Axl tyrosine kinase inhibitor, which can act by inhibiting the initial autophosphorylation event and/or by inhibiting the phosphorylation of a protein substrate, for example, by competing with the protein substrate or ATP for sterically binding with Axl. An Axl protein inhibitor can also act by more than one of these mechanisms. Axl modulators include nonproteinaceous modulators, for example, small molecules and nucleic acids, including interfering RNAs, as well as peptides, antibodies, and other proteins (including those that bind to Axl), as well as modified forms or fragments thereof, propeptides, peptides, and mimetics of all of these modulators.

### Small Molecules

Modulators of Axl protein activity useful in the methods of the invention to treat or prevent cartilage and bone disorders include small molecules and compounds. Small molecule inhibitors of Axl protein activity can directly inhibit tyrosine phosphorylation by physical interactions with the highly conserved kinase domain, by binding the substrate-binding site and/or the ATP binding site. Compounds that bind both the ATP and protein substrate binding sites are sometimes referred to as competitive bisubstrate inhibitors. Small molecules include synthetic and purified naturally occurring Axl protein activity modulators. Small molecules can be mimetics or secretagogues. Small molecules that inhibit Axl protein kinase activity are described, e.g., in U.S. Patent Publication No. 2007/0142402.

### Nucleic Acids

Axl modulators useful in the methods of the invention to treat or prevent bone disorders include nucleic acids. The terms “polynucleotide,” “oligonucleotide,” and “nucleic acid” refer to deoxyribonucleic acid (DNA) and, where appropriate, to ribonucleic acid (RNA), or peptide nucleic acid (PNA). The term should also be understood to include nucleotide analogs, and single or double stranded polynucleotides (e.g., siRNA). Examples of polynucleotides include, but are not limited to, plasmid DNA or fragments thereof, viral DNA or RNA, RNAi, etc.

Nucleic acids that that can block the Axl protein activity are useful in this invention. Such inhibitors may encode proteins that interact with Axl protein itself. Alternatively, such inhibitors may encode proteins that can interact with a protein interacting with the Axl protein (such as Gas6). Inhibitors may also encode proteins that interact with both Axl and an interacting protein.

The methods of the invention can include the use of RNA interference (“RNAi”) to reduce the expression of Axl. RNAi can be initiated by introducing nucleic acid molecules, e.g. synthetic short interfering RNAs (“siRNAs”) or RNA interfering agents, to inhibit or silence the expression of target genes. See, for example, U.S. Patent Publication No. 20030153519, and U.S. Pat. Nos. 6,506,559, 6,573,099, and 7,144,706.

An “RNA interfering agent” or “RNAi” as used herein is any agent that interferes with or inhibits expression of a target gene or genomic sequence by RNA interference. Such RNA interfering agents include, but are not limited to, RNA molecules which are homologous to the target gene or genomic sequence, or a fragment thereof, short interfering RNA (siRNA), short hairpin or small hairpin RNA (shRNA), and small molecules which interfere with or inhibit expression of a target gene by RNA interference.

As used herein, “inhibition of target gene expression” includes any decrease in the level of expression of the target gene or the level of protein encoded by the target gene. The decrease may be of at least 30%, 40%, 50%, 60%, 70%, 80%, 90%, 95%, 99%, or more as compared to the expression of a target gene or the activity or level of the protein encoded by a target gene that has not been targeted by an RNA interfering agent. siRNAs have a well defined structure. They are normally a short double-strand of RNA (dsRNA) with 2-nt 3′ overhangs on either end. An siRNA may be chemically synthesized, may be produced by in vitro transcription, or may be produced within a host cell. Typically, an siRNA is at least 15-50 nucleotides long, e.g., 20, 21, 22, 23, 24, 25, 26, 27, 28, 29, or 30 nucleotides in length, or any integer thereof. The siRNA is a double stranded RNA (dsRNA) of about 15 to about 40 nucleotides in length, for example, about 15 to about 28 nucleotides in length, including about 19, 20, 21, or 22 nucleotides in length, and may contain a 3′ and/or 5′ overhang on each strand having a length of about 0, 1, 2, 3, 4, 5, or 6 nucleotides. The siRNA can inhibit a target gene by transcriptional silencing. The siRNA is capable of promoting RNA interference through degradation or specific post-transcriptional gene silencing (PTGS) of the target messenger RNA.

RNA is useful in the methods of the invention also include small hairpin RNAs (shRNAs). shRNAs are composed of a short (e.g. about 19 to about 25 nucleotide) antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids and viral vectors.

The targeted region of the siRNA molecules of the present invention can be selected from a given target sequence. For example, nucleotide sequences can begin from about 25-100 nucleotides downstream of the start codon. Nucleotide sequences can contain 5′ or 3′ untranslated regions, as well as regions near the start codon. Methods for the design and preparation of siRNA molecules are well known in the art, including a variety of rules for selecting sequences as RNAi reagents (see, e.g., Boese et al., Methods Enzymol. 392:73-96 (2005)).

siRNA may be produced using standard techniques as described in Hannon, Nature 418:244-251 (2002); McManus et al., Nat. Reviews 3:737-747 (2002); Heasman, Dev. Biol. 243:209-214 (2002); Stein, J. Clin. Invest. 108:641-644 (2001); and Zamore, Nat. Struct. Biol., 8:746-750 (2001). Preferred siRNAs are 5-prime phosphorylated. Such siRNAs can be custom developed though multiple Web sites including, but not limited to, those provided by companies such as Dharmacon (Lafayette, Colo.), Invitrogen (Carlsbad, Calif.), Qiagen (Valencia, Calif.), and Ambion (Austin, Tex.). The siRNA sequences described herein (SEQ ID NOs:3, 4, 5, and 6) were purchased from Dharmacon.

Additional RNAi constructs were developed internally using standard techniques. Constructs developed include shRNAs specific to human, which are hAxl 363, hAxl 1107, hAxl 1748, hAxl 1988, and hAxl 2448, and shRNAs specific to mouse, which are mAxl 187, mAxl 1079, mAxl 1477, mAxl 1850, and mAxl 2269. These shRNAs were generated using plasmids comprising the DNA sequences set forth in SEQ ID NOs: 18, 19, 20, 21, 22, 23, 24, 25, 26, and 27 driven by the U6 promoter.

The nucleotide sequence set forth in SEQ ID NO:18 comprises hAxl 363 and is shown below:

5′-CGGACATCAGACCTTCGTGTCTTCCTGTCAACACGAAGGTCTGATGT
CC-3′

The nucleotide sequence set forth in SEQ ID NO:19 comprises hAxl 1107 and is shown below:

5′-CGCGTATCAAGGCCAGGACACTTCCTGTCATGTCCTGGCCTTGATAC
GC-3′

The nucleotide sequence set forth in SEQ ID NO:20 comprises hAxl 1748 and is shown below:

5′-CGAGTGAAGCGGTCTGCATGCTTCCTGTCACATGCAGACCGCTTCAC
TC-3′

The nucleotide sequence set forth in SEQ ID NO:21 comprises hAxl 1988 and is shown below:

5′-CGAGTACCAAGAGATTCATACTTCCTGTCATATGAATCTCTTGGTAC
TC-3′

The nucleotide sequence set forth in SEQ ID NO:22 comprises hAxl 2448 and is shown below:

5′-CGACGAAATCCTCTATGTCACTTCCTGTCATGACATAGAGGATTTCG
TC-3′

The nucleotide sequence set forth in SEQ ID NO:23 comprises mAxl 187 and is shown below:

5′-CGGCTTCGAGATGGACAGATCTTCCTGTCAATCTGTCCATCTCGAAG
CC-3′

The nucleotide sequence set forth in SEQ ID NO:24 comprises mAxl 1079 and is shown below:

5′-CGTACCGGCTGGCATATCGACTTCCTGTCATCGATATGCCAGCCGGT
AC-3′,

The nucleotide sequence set forth in SEQ ID NO:25 comprises mAxl 1477 and is shown below:

5′-CGTGTCCGAAAGTCCTACAGCTTCCTGTCACTGTAGGACTTTCGGAC
AC-3′

The nucleotide sequence set forth in SEQ ID NO:26 comprises mAxl 1850 and is shown below:

5′-CGAAACACGGAGACCTACACCTTCCTGTCAGTGTAGGTCTCCGTGTT
TC-3′

The nucleotide sequence set forth in SEQ ID NO:27 comprises mAxl 2269 and is shown below:

5′-CGTCAAGGAAATCGGCTGAACTTCCTGTCATTCAGCCGATTTCCTTG
AC-3′

Axl gene expression has been targeted using siRNA inhibitors. The sequences of four Axl-specific siRNAs are provided in SEQ ID NOs:3-6. Other Axl siRNA and shRNAs have been reported in the literature. To test if knockdown expression of Axl in the L929 subline, that is resistant to TNFαα-induced cell death, would restore sensitivity to TNFα, Budagian et al. used siRNAs to decrease the levels of Axl mRNA and Axl protein (EMBO J., 24:4260-4270 (2005)) The siRNAs used by Budagian et al. have the sequence set forth here in SEQ ID NO:28, SEQ ID NO:29, and SEQ ID NO:30, and shown below:

| | |
|---|---|
| SEQ ID NO:28 | 5′-UAUCACAGGUGCCAGAGGA-3′ |
| SEQ ID NO:29 | 5′-AAGACAUCCUCUUUCUCCUGC-3′ |
| SEQ ID NO:30 | 5′-AAGAUUUGGAGAACACACUGA-3′ |

Using shRNAs to knockdown Axl, Holland, et al. (Cancer Res. 65:9294-9303 (2005)) show that Axl is necessary for ex vivo angiogenesis in a mouse model. The shRNAs reported by Holland et al. have the sequences set forth in SEQ ID NO:31 and SEQ ID NO:32 and are shown below:

SEQ ID NO:31:
5′-GACATCCTCTTTCTCCTGCGAAGCCCATGAAGCTTGATGGGCTTCGC
AGGAGAAAGAGGATGTC-3′
SEQ ID NO:32:
5′-GATTTGGAGAACACACTGAAGGCCTTGCGAAGCTTGGCAAGGCCTTC
AGTGTGTTCTCCAAATC-3′

Shieh et al., Neoplasia 7:1058-1064 (2005) describes studies using Axl siRNAs but does not reveal the sequences of the siRNAs used. In this publication transfection of the Cl1-5 cell line using four pooled siRNA duplexes resulted in a knockdown of Axl RNA and Axl protein, as indicated by PCR and Western blot analyses.

Antisense oligonucleotides can be used to reduce the expression of Axl. “Antisense,” as used herein, refers to a nucleic acid capable of hybridizing to a portion of a coding and/or noncoding region of mRNA by virtue of sequence complementarity, thereby interfering with translation from the mRNA. The antisense oligonucleotides may be either DNA or RNA fragments. Antisense polynucleotides may be produced using standard techniques, as described in Antisense Drug Technology: Principles, Strategies, and Applications, 1st ed., Ed. Crooke, Marcel Dekker (2001).

Nucleic acids may be administered at a dosage from about 1 μg/kg to about 20 mg/kg, depending on the severity of the symptoms and the progression of the disorder. The appropriate effective dose is selected by a treating clinician from the following ranges: about 1 μg/kg to about 20 mg/kg, about 1 μg/kg to about 10 mg/kg, about 1 μg/kg to about 1 mg/kg, about 10 μg/kg to about 1 mg/kg, about 10 μg/kg to about 100 μg/kg, about 100 μg to about 1 mg/kg, and about 500 μg/kg to about 1 mg/kg. Nucleic acid inhibitors may be administered via topical, oral, intravenous, intraperitoneal, intramuscular, intracavity, subcutaneous or transdermal means.

The nucleic acids may be obtained, isolated, and/or purified from their natural environment, in substantially pure or homogeneous form. Systems for the manipulation of nucleic acids, including cloning and gene expression in a variety of different host cells and systems are well known, and described in detail in Short Protocols in Molecular Biology, Eds. Ausubel et al., 5^{th} ed., John Wiley & Sons (2002). Suitable vectors can be chosen or constructed, containing appropriate regulatory sequences using methods well known in the art.

See, e.g., Molecular Cloning: A Laboratory Manual, Sambrook et al., 3^{rd} ed., Cold Spring Harbor Laboratory Press (2001).

A nucleic acid can be fused to other sequences encoding additional polypeptide sequences, for example, sequences that function as a marker or reporter. Examples of marker or reporter genes include—lactamase, chloramphenicol acetyltransferase (CAT), adenosine deaminase (ADA), aminoglycoside phosphotransferase (responsible for neomycin (G418) resistance), dihydrofolate reductase (DHFR), hygromycin-B-phosphotransferase (HPH), thymidine kinase (TK), lacZ (encoding-galactosidase), xanthine guanine phosphoribosyltransferase (XGPRT), luciferase, and many others known in the art.

### Protein Modulators

Proteins that bind to Axl and change its activity are acceptable modulators for use in the methods of the invention.

### Peptides

Nonphosphorylatable peptides that interact with the intracellular substrate-binding region of Axl and inhibit its tyrosine kinase activity can be used as inhibitors in the methods of the invention. Such peptides, sometimes referred to as substrate inhibitors or pseudosubstrates, are short peptides designed to mimic the primary sequence around the substrate's tyrosine moiety, and typically substitute nonphosphorylatable tyrosine analogues such as phenylalanine, tyramine, or iodotyrosine for the tyrosine moiety. For example, the peptides having the amino acid sequences set forth in SEQ ID NO:14 and SEQ ID NO:15 have been identified as Axl autophosphorylation sites (i.e. they are Axl substrates), and can provide the basis for generating substrate inhibitors or pseudosubstrates: These Axl-specific substrates were identified based on their homology to putative autophosphorylation sites in the closely-related Axl family member, Mer (Ling et al., J. Biol. Chem. 271:18355-62 (1996)). The amino acid sequences set forth in SEQ ID NO:14 and SEQ ID NO:15 are shown below:

| | |
|---|---|
| SEQ ID NO:14: | KQPADCLDGLYALMSRCWELN |
| SEQ ID NO:15 | FGLSKKIYNGDYYRQGRIAK |

### Antibodies

Antibodies that regulate the activity of Axl protein can be used in the methods of the invention.

The term “antibody,” as used herein, refers to an immunoglobulin or a part thereof, and encompasses any polypeptide comprising an antigen-binding site regardless of the source, species of origin, method of production, and characteristics. As a non-limiting example, the term “antibody” includes human, orangutan, mouse, rat, goat, sheep, and chicken antibodies. The term includes but is not limited to polyclonal, monoclonal, monospecific, polyspecific, non-specific, humanized, single-chain, chimeric, synthetic, recombinant, hybrid, mutated, and CDR-grafted antibodies, as well as intrabodies. For the purposes of the present invention, it also includes, unless otherwise stated, antibody fragments such as Fab, F(ab′)₂, Fv, scFv, Fd, dAb, and other antibody fragments that retain the antigen-binding function.

According to the methods described above, antibodies can be developed that specifically bind to the Axl protein itself. As described above, amino acid sequence of Axl is provided in SEQ ID NO:2, as well as Genbank Accession No. NP_{—}068713. The amino acid sequence of Axl isoform 2 is found in Genbank Accession No. NP_{—}001690. Antibodies that are most effective in this invention will have the property of binding specifically to Axl protein. Specifically, GAS6:Axl crystal structure information (Sasaki, et al. EMBO J. 25:80-87 (2006)) on their interaction has demonstrated that the two IG domains at the extracellular region of the Axl molecule designated IG1 and IG2 are both involved in Gas6 interaction. However, the major contact of GAS6 with Axl is located on the IG1 region which is referred to herein as “the major binding site” and has the amino acid sequence set forth in SEQ ID NO: 33. There is a minor contact site on the IG2 region, which is referred to herein as “the minor binding site” and has the amino acid sequence set forth in SEQ ID NO:34. The sequences of the Axl IG1 and IG2 regions are shown below with the Gas6 contact sites shown bolded and underlined:

SEQ ID NO:33:
TLRCQLQVQGEPPEVHWLRDGQILELADSTQTQVPLGEDEQDDWIVVSQL
RITSLQLSDTGQYQCLVFLGHQTFVSQPGYVGLE
SEQ ID NO:34
GLPYFLEEPEDRTVAANTPFNLSCQAQGPPEPVDLLWLQDAVPLATAPGH
GPQRSLHVPGLNKTSSFSCEAHNAKGVTTSRTATIT

Importantly, since Axl protein activation by Gas6 requires interaction at both the major and the minor binding sites, an antibody directed to one site may be sufficient to block Gas6 interaction with Axl. The amino acid sequence of IG2, the minor binding site of Gas6 on RTK, is highly conserved between Axl, Tyro3, and Mer, whereas there is less conservation in the amino acid sequence of IG1, the major binding site of Gas6 on RTK. Therefore, an Axl nucleotide sequence to target would be the contact points of Gas6 (and/or neighboring sequences) in the IG1 domain of Axl protein.

Antibodies can be developed against the whole receptor protein, or against only the extracellular domain. Antibodies can also be developed against an intracellular epitope of Axl, for example the kinase domain. Antibodies may be developed against variants and fragments of Axl. Antibodies can be raised against a soluble dimeric form of the extracellular domain of Axl (U.S. Patent Publication No. 20050147612).

Such antibodies may be capable of binding Axl with high affinity, and may bind the mature protein in monomeric form, homodimer form, and/or heterodimer form. These antibodies will be effective in the invention if they inhibit an activity of Axl. Antibody binding to Axl can block the kinase activity of the Axl receptor. Antibody binding to Axl can also block binding of Axl to its ligand, such as Gas6; such antibodies can also block the Axl kinase activity. Antibody binding to Axl can block its dimerization. As described above, Axl dimers include homodimers of the full length, membrane bound protein as well as sAxl-sAxl homodimers, Axl-sAxl heterodimers, Axl-Gas6 heterodimers, and sAxl-Gas6 heterodimers.

Antibodies against Axl have been described in the art and are contemplated for use in the invention. Examples of such antibodies include, but are not limited to, for example, antibodies set forth in U.S. Pat. No. 6,191,261; and O'Bryan et al., J. Biol. Chem. 270:551-557 (1995)). Commercially available antibodies are also available, including human polyclonal antibodies and several murine monoclonal antibodies (R+D Systems, Minneapolis, Minn.).

The invention provides neutralizing antibodies against Axl. The term “neutralizing antibody,” as used herein, refers to an antibody having the antigen binding site to a specific receptor capable of reducing or inhibiting (i.e., blocking) activity or signaling through an Axl receptor. Such antibodies typically block ligand-dependent activation and/or constitutive, ligand-independent activation of Axl. Neutralizing antibodies for Axl have been described in the art and are contemplated for use in the present invention, including the commercially available goat anti-human Axl polyclonal antibody from R&D Systems (Catalog No. AF154).

Small modular immunopharmaceutical products (SMIP™ products) are a highly modular compound class having enhanced drug properties over monoclonal and recombinant antibodies. SMIP™ products comprise a single polypeptide chain including a target-specific binding domain, based, for example, upon an antibody variable domain, in combination with a variable FC region that permits the specific recruitment of a desired class of effector cells (such as, e.g., macrophages and natural killer (NK) cells) and/or recruitment of complement-mediated killing. Depending upon the choice of target and hinge regions, SMIP™ products can signal or block signalling via cell surface receptors.

### Modified Axl Receptors

Modified Axl proteins that inhibit the activity of unmodified Axl receptors may be used in the methods of the invention. Such modified receptors are sometimes referred to as dominant negative receptors, because these variants adversely affect the normal, wild-type gene product within the same cell. A modified Axl receptor can interact with an Axl ligand, inhibiting the ligand's activity or binding to its receptor, i.e. the unmodified Axl receptor. Alternatively, modified Axl receptors can interact directly with Axl receptor (i.e. unmodified Axl receptors). Such modified Axl receptors may bind Axl in monomeric form, homodimer form, and/or heterodimer form. Modified Axl receptors, of course, may interact with both Axl ligand and its receptor. Modified Axl receptors include soluble Axl receptors, dominant negative Axl receptors, and kinase dead Axl receptors.

Modified soluble Axl receptors can be used in the methods of the invention. Soluble receptors may comprise all or part of the extracellular domain (also referred to as the ectodomain) of Axl. The modified soluble receptors bind an Axl ligand, including Gas6, reducing the ability of the Axl ligand to bind to its native receptor(s) in the body. The modified soluble receptors can block dimerization of the unmodified Axl. Soluble receptors may be produced recombinantly or by chemical or enzymatic cleavage of the intact receptor.

Several modified soluble Axl receptors have been described. For example, Nagata et al. described a truncated Axl receptor which contains the first 438 amino acids of Axl fused to amino acids 216-443 of human IgG1 via a 5 amino acid linker (J. Biol. Chem. 271 (47):30022-30027 (1996)). An Axl extracellular domain-Fc fusion protein which inhibits Axl, consisting of the Axl ectodomain fused to a spacer with the sequence Gly-Pro-Gly, followed by the hinge CH2 and CH3 regions of human IgG1, is set forth in Shankar et al., J. Neurosci. 23:4208-4218 (2003). U.S. Patent Publication No. 20050186571 describes a truncated Axl protein comprising the Axl extracellular domain, referred to as a dominant negative variant, generated by subcloning the 1.5 kb EcoRI/FspI fragment of the cDNA sequence.

A human Axl/Fc fusion protein is commercially available from R+D Systems (Minneapolis, Minn.). This Axl/Fc chimera contains the extracellular domain of human Axl (amino acids 1-442) fused via a 7 amino acid linker to the carboxy-terminal 6× histidine-tagged Fc region of human IgG1. The recombinant mature human Axl/Fc is a disulfide linked homodimeric protein. Based on N-terminal sequencing, the protein begins with Glu26. The reduced human Axl/Fc monomer has a calculated molecular mass of 72.3 kDa. As a result of glycosylation, the recombinant Axl monomer migrates as an approximately 100-110 kDa protein in SDS-PAGE under reducing conditions.

A mouse Axl/Fc fusion protein is also commercially available from R+D Systems (Minneapolis, Minn.). This Axl/Fc chimera has contains the extracellular domain of mouse Axl (amino acids 1-443) fused via a 6 amino acid linker to the carboxy-terminal 6× histidine-tagged Fc region of human IgG1. The recombinant mature human Axl/Fc is a disulfide linked homodimeric protein. Based on N-terminal sequencing, the protein begins with His20. The reduced human Axl/Fc monomer has a calculated molecular mass of 73.8 kDa. As a result of glycosylation, the recombinant Axl monomer migrates as an approximately 100-110 kDa protein in SDS-PAGE under reducing conditions.

Modified Axl proteins that have inactive kinase domains may be used in the methods of the invention; these variants are also referred to as “kinase dead” Axl receptors. To generate a kinase dead Axl receptor, a point mutation can be introduced to affect a residue essential to the kinase activity, such as ablating the conserved ATP-binding lysine residue in the tyrosine kinase domain, resulting in its inability to phosphorylate its substrates. For example, a kinase dead Axl receptor has been described, which has a substitution of Arg for Lys at amino acid position 567 (McCloskey et al., J. Biol. Chem. 272:23285-23291 (1997); Fridell et al., J. Biol. Chem. 273:7123-7126 (1998)).

An Axl protease can be used in the methods of the invention. Axl activity can be downregulated by administration of a protease that cleaves the extracellular domain (ECD) of the Axl receptor. This cleavage is an in vivo phenomenon that modulates the Gas6 function at two levels (O'Bryan et al., J. Biol. Chem. 270:551-557 (1995)). The released Axl-ECD will bind to Gas6 and prevent signaling of Gas6. The membrane-bound intracellular domain of Axl retains its kinase activity, but is quickly degraded. The cleavage site in the Axl sequence has been mapped to a peptide of 14 amino acids (VKEPSTPAFSWPWW (SEQ ID NO:12) which is amino-terminal to the transmembrane region. A high dose will strip the cell of its Axl-ECD, therefore part of the Gas6 protein will be scavenged and the Axl receptor will be degraded.

Axl proteinaceous inhibitors are optionally glycosylated, pegylated, or linked to another nonproteinaceous polymer. Inhibitors of Axl may be modified to have an altered glycosylation pattern (i.e., altered from the original or native glycosylation pattern). As used herein, an “altered glycosylation pattern” means having one or more carbohydrate moieties added or deleted, and/or having one or more glycosylation sites added or deleted as compared to the original inhibitor. Addition of glycosylation sites to the inhibitors may be accomplished by altering the amino acid sequence to contain glycosylation site consensus sequences well known in the art. Another means of increasing the number of carbohydrate moieties is by chemical or enzymatic coupling of glycosides to the amino acid residues of the inhibitor. These methods are described in WO 87/05330, and in Aplin et al., Crit. Rev. Biochem. 22:259-306 (1981). Removal of any carbohydrate moieties present on the receptor may be accomplished chemically or enzymatically as described by HakimuddinSojar et al., Arch. Biochem. Biophys. 259:52-57 (1987); Edge et al., Anal. Biochem. 118:131-137 (1981); and by Thotakura et al., Meth. Enzymol. 138:350-359 (1987).

The Axl inhibitors useful in the methods of the invention may also be tagged with a detectable or functional label. Detectable labels include radiolabels such as ¹²⁵I, ¹³¹I or ⁹⁹Tc, which may be attached to the inhibitors using conventional chemistry known in the art. Labels also include enzyme labels such as horseradish peroxidase or alkaline phosphatase. Labels further include chemical moieties such as biotin, which may be detected via binding to a specific cognate detectable moiety, e.g., labeled avidin.

Any of the proteins that bind to Axl can be made more stable by fusion to another protein or portion of another protein. Increased stability is advantageous for therapeutics as they can be administered at a lower dose or at less frequent intervals. Fusion to at least a portion of an immunoglobulin, such as the constant region, optionally an Fc fragment of an immunoglobulin, can increase the stability of these proteins. The preparation of such fusion proteins is well known in the art and can be performed easily. See, e.g., Spiekermann et al. J. Exp. Med., 196:303-310 (2002).

Mimetics of Axl inhibitors, including peptide inhibitors, antibodies, and other protein inhibitors, may be used in the methods of the invention. Any synthetic analogue of these Axl inhibitors, especially those with improved in vitro characteristics such as having a longer half-life, or being less easily degraded by the digestive system, are useful. Mimetics will be effective in the methods of the invention if they block the activity of Axl. Mimetics that are most effective in this invention will have the property of binding specifically to Axl, and may inhibit Axl activity in vitro and in vivo.

### Methods for Identifying Axl Modulators

The invention provides any one or more methods to identify compounds which modulate bone growth, by contacting a cell with a test compound, and determining whether the musculoskeletal activity or expression of Axl by the cell is changed as a result of the presence of the test compound. An increase in the musculoskeletal activity or expression of Axl indicates that the compound negatively affects bone growth; such a compound is useful for the prevention or treatment of disorders characterized by excessive bone. A decrease in the musculoskeletal activity or expression of Axl indicates that the compound positively effects bone growth; such a compound is useful for the prevention or treatment of bone degenerative disorders. The compounds are pre-screened to determine whether the test compound changes the tyrosine kinase activity of Axl.

Interactions of small molecules or peptides with Axl can be analyzed in real time using Biacore systems technology (Biacore International AB, Uppsala, Sweden). The invention also contemplates the use of additional screening assays, e.g. secondary and tertiary assays, to further identify the effect of such molecules on bone cell differentiation and function, and on bone density, for example, using assays described in detail above.

### Cells

Any cells that express Axl can be used in assays to identify test compounds that modulate bone growth. For example, useful cells include, but are not limited to, osteoblasts, osteoblast precursors, mesenchymal stem cells, osteoprogenitor cells derived from bone marrow, and osteoprogenitor cells circulating in blood. Useful in practicing the methods of the invention are skeletal bone cells including osteoprogenitor cells, bone lining cells, osteoblasts, osteocytes. Cell types that may also be used include embryonic fibroblasts, myoblastic precursors or adipocyte lineage (which would include pre-adipocyte). Immortalized or transformed cells may be used in vitro to evaluate the activity of a compound or therapeutic agent as a modulator of Axl gene expression or protein activity before testing the compound or therapeutic agent in vivo animal models. Useful cells also include endochondral skeletal progenitor cells derived from mouse limb bud, referred to as the cell line “Clone 14.” See Rosen et al., J. Bone Miner. Res. 9:1759-1768 (1994). Useful cells may also be obtained from the American Tissue Culture Collection (ATCC) and include, among others, MC3T3-E1 cells, embryonic fibroblasts, such as C3H10T½ cells myoblastic precursor cells, such as C2C12 cells, and pre-adipocytes, such as 3T3-LI. Other suitable cell lines are well known to persons of skill in the art. In another example, the method employs suitable animals such as mammals including, but not limited to, rats, rabbits, sheep, pigs, dogs, cats, monkeys, chimpanzees, and guinea pigs. In a particular example, the animal is a rodent, e.g., a mouse.

### Test Compounds

The methods and assays of the invention can be used to screen panels of test compounds or to confirm the inhibitory or stimulatory activity of a known bone growth modulator. The test compound may be part of a library of compounds of interest, or it may be part of a library of structurally-related compounds. The structure of the compound may be known or unknown. Test compounds may be predetermined by known functions or structures. For example, a test compound may be chosen because it effects the tyrosine kinase activity of Axl or another receptor tyrosine kinase. Similarly, a test compound may be selected because of its homology to a known Axl modulator. Alternatively, selection of the test compound can be arbitrary. In non-limiting examples, the test compound may be a peptide, a protein or protein fragment, a small organic molecule, a chemical composition, a nucleic acid, an aptamer, or an antibody. A number of methods for evaluating the appropriateness of a test compound are well known.

The test compound may be part of a larger scale screening of compounds. The test compound can be pre-selected or pre-screened to identify test compounds that alter the tyrosine kinase activity of Axl. As described above, small molecule inhibitors of Axl can directly inhibit tyrosine phosphorylation by binding the substrate binding site and/or the ATP binding site in the intracellular kinase domain. Methods to identify small molecule tyrosine kinase inhibitors (TKIs) for receptor tyrosine kinases are well known and have been generally identified using one of the following strategies: mimicking the structure of known natural kinase inhibitors, molecular modeling of the kinase domain, and large scale screening methods. U.S. Patent Publication No. 20070142402 (herein incorporated by reference in its entirety) describes the identification and use of small molecule compounds that inhibit Axl kinase activity. Such compounds can be used in the methods of the invention described herein.

### Kinases

Polynucleotide fragments that encode polypeptides that exhibit Axl kinase activity are useful in the methods of the invention. Such polypeptides include those having the amino acid sequence set forth in SEQ ID NO:13, which is shown below:

SEQ ID NO: 13:
MGHHHHHHRRKKETRYGEVFEPTVERGELVVRYRVRKSYSRRTTEATLNS
LGISEELKEKLRDVMVDRHKVALGKTLGEGEFGAVMEGQLNQDDSILKVA
VKTMKIAICTRSELEDFLSEAVCMKEFDHPNVMRLIGVCFQGSERESFPA
PVVILPFMKHGDLHSFLLYSRLGDQPVYLPTQMLVKFMADIASGMEYLST
KRFIHRDLAARNCMLNENMSVCVADFGLSKKIYNGDYYRQGRIAKMPVKW
IAIESLADRVYTSKSDVWSFGVTMWEIATRGQTPYPGVENSEIYDYLRQG
NRLKQPADCLDGLYALMSRCWELNPQDRPSFAELREDLENTLKALPPAQE
PDEILYVNMDEGGGYPEPPGAAGGADPPTQPDPKDSCSCLTAAEVHPAGR
YVLCPSTTPSPAQPADRGSPAAPGQEDGA

Other polypeptides that exhibit Axl kinase activity include those whose amino acid sequence is set forth in SEQ ID NO:37, SEQ ID NO:38, SEQ ID NO:39, SEQ ID NO:40, SEQ ID NO:41, and SEQ ID NO:42, and are shown below:

SEQ ID NO:37:
MGRRKKETRYGEVFEPTVERGELVVRYRVRKSYSRRTTEATLNSLGISEE
LKEKLRDVMVDRHKVALGKTLGEGEFGAVMEGQLNQDDSILKVAVKTMKI
AICTRSELEDFLSEAVCMKEFDHPNVMRLIGVCFQGSERESFPAPVVILP
FMKHGDLHSFLLYSRLGDQPVYLPTQMLVKFMADIASGMEYLSTKRFIHR
DLAARNCMLNENMSVCVADFGLSKKIYNGDYYRQGRIAKMPVKWIAIESL
ADRVYTSKSDVWSFGVTMWEIATRGQTPYPGVENSEIYDYLRQGNRLKQP
ADCLDGLYALMSRCWELNPQDRPSFAELREDLENTLKALPPAQEPDEILY
VNMDEGGGYPEPPGAAGGADPPTQPDPKDSCSCLTAAEVHPAGRYVLCPS
TTPSPAQPADRGSPAAPGQEDGAHHHHHH
SEQ ID NO:38:
ELVVRYRVRKSYSRRTTEATLNSLGISEELKEKLRDVMVDRHKVALGKTL
GEGEFGAVMEGQLNQDDSILKVAVKTMKIAICTRSELEDFLSEAVCMKEF
DHPNVMRLIGVCFQGSERESFPAPVVILPFMKHGDLHSFLLYSRLGDQPV
YLPTQMLVKFMADIASGMEYLSTKRFIHRDLAARNCMLNENMSVCVADFG
LSKKIYNGDYYRQGRIAKMPVKWIAIESLADRVYTSKSDVWSFGVTMWEI
ATRGQTPYPGVENSEIYDYLRQGNRLKQPADCLDGLYALMSRCWELNPQD
RPSFAELREDLENTLKALPPAQEPDEILYVNMDEGGGYPEPPGAAGGADP
PTQPDPKDSCSCLTAAEVHPAGRYVLCPSTTPSPAQPADRGSPAAPGQED
GA
SEQ ID NO:39:
EATLNSLGISEELKEKLRDVMVDRHKVALGKTLGEGEFGAVMEGQLNQDD
SILKVAVKTMKIAICTRSELEDFLSEAVCMKEFDHPNVMRLIGVCFQGSE
RESFPAPVVILPFMKHGDLHSFLLYSRLGDQPVYLPTQMLVKFMADIASG
MEYLSTKRFIHRDLAARNCMLNENMSVCVADFGLSKKIYNGDYYRQGRIA
KMPVKWIAIESLADRVYTSKSDVWSFGVTMWEIATRGQTPYPGVENSEIY
DYLRQGNRLKQPADCLDGLYALMSRCWELNPQDRPSFAELREDLENTLKA
LPPAQEPDEILYVNMDEGGGYPEPPGAAGGADPPTQPDPKDSCSCLTAAE
VHPAGRYVLCPSTTPSPAQPADRGSPAAPGQEDGA
SEQ ID NO:40:
DVMVDRHKVALGKTLGEGEFGAVMEGQLNQDDSILKVAVKTMKIAICTRS
ELEDFLSEAVCMKEFDHPNVMRLIGVCFQGSERESFPAPVVILPFMKHGD
LHSFLLYSRLGDQPVYLPTQMLVKFMADIASGMEYLSTKRFIHRDLAARN
CMLNENMSVCVADFGLSKKIYNGDYYRQGRIAKMPVKWIAIESLADRVYT
SKSDVWSFGVTMWEIATRGQTPYPGVENSEIYDYLRQGNRLKQPADCLDG
LYALMSRCWELNPQDRPSFAELREDLENTLKALPPAQEPDEILYVNMDEG
GGYPEPPGAAGGADPPTQPDPKDSCSCLTAAEVHPAGRYVLCPSTTPSPA
QPADRGSPAAPGQEDGA
SEQ ID NO:41:
MGRRKKETRYGEVFEPTVERGELVVRYRVRKSYSRRTTEATLNSLGISEE
LKEKLRDVMVDRHKVALGKTLGEGEFGAVMEGQLNQDDSILKVAVKTMKI
AICTRSELEDFLSEAVCMKEFDHPNVMRLIGVCFQGSERESFPAPVVILP
FMKHGDLHSFLLYSRLGDQPVYLPTQMLVKFMADIASGMEYLSTKRFIHR
DLAARNCMLNENMSVCVADFGLSKKIYNGDYYRQGRIAKMPVKWIAIESL
ADRVYTSKSDVWSFGVTMWEIATRGQTPYPGVENSEIYDYLRQGNRLKQP
ADCLDGLYALMSRCWELNPQDRPSFAELREDLENTLKALPPAQEPDEILY
VNMD
SEQ ID NO:42:
MGRRKKETRYGEVFEPTVERGELVVRYRVRKSYSRRTTEATLNSLGISEE
LKEKLRDVMVDRHKVALGKTLGEGEFGAVMEGQLNQDDSILKVAVKTMKI
AICTRSELEDFLSEAVCMKEFDHPNVMRLIGVCFQGSERESFPAPVVILP
FMKHGDLHSFLLYSRLGDQPVYLPTQMLVKFMADIASGMEYLSTKRFIHR
DLAARNCMLNENMSVCVADFGLSKKIYNGDYYRQGRIAKMPVKWIAIESL
ADRVYTSKSDVWSFGVTMWEIATRGQTPYPGVENSEIYDYLRQGNRLKQP
ADCLDGLYALMSRCWELNPQDRPSFAELREDLENTLK

Another polypeptide that exhibits Axl kinase activity has the amino acid sequence set forth in SEQ ID NO:43, and shown below:

MHRRKKETRYGEVFEPTVERGELVVRYRVRKSYSRRTTEATLNSLGISEE
LKEKLRDVMVDRHKVALGKTLGEGEFGAVMEGQLNQDDSILKVAVKTMKI
AICTRSELEDFLSEAVCMKEFDHPNVMRLIGVCFQGSERESFPAPVVILP
FMKHGDLHSFLLYSRLGDQPVYLPTQMLVKFMADIASGMEYLSTKRFIHR
DLAARNCMLNENMSVCVADFGLSKKIYNGDYYRQGRIAKMPVKWIAIESL
ADRVYTSKSDVWSFGVTMWEIATRGQTPYPGVENSEIYDYLRQGNRLKQP
ADCLDGLYALMSRCWELNPQDRPSFAELREDLENTLKALPPAQEPDEILY
VNMDEGGGYPEPPGAAGGADPPTQPDPKDSCSCLTAAEVHPAGRYVLCPS
TTPSPAQPADRGSPAAPGQEDGAGHHHHHH

Kinase activity assays which are particularly well-suited for prescreening to identify test compounds that alter the tyrosine kinase activity of Axl include Time Resolved Fluorescence Resonance Energy Transfer (TR-FRET) assays such as Lanthascreen™, AlphaScreen®, or Lance™-type assays. Lanthascreen™ (Invitrogen, Carlsbad, Calif.) can be used to directly measure the ability of the Axl kinase domain to phosphorylate substrate. This assay uses a Terbium-labeled anti-phosphotyrosine antibody (donor) to detect phosphorylation of a fluorescein-labeled substrate (acceptor). When these two labels are brought into close proximity (i.e. when the antibody recognizes phosphorylated substrate), fluorescence energy transfer occurs, resulting in an increase in acceptor fluorescence and a decrease in donor fluorescence. Examples of peptides which can be used in a Lanthascreen™ assay include: 5-FAM-DCLDGLYALMSRC (the amino acid sequence of which is set forth in SEQ ID NO:16) and 5-FAM-KKIYNGDYYRQG (the amino acid sequence of which is set forth in SEQ ID NO:17). “5-FAM” refers to the conjugation of 5-carboxyfluorescein to the amino terminus of the peptide. Methods and reagents for accomplishing such conjugation are commercially available (e.g. AnaTag™ 5-FAM protein labeling kit, AnaSpec, San Jose, Calif.). Other peptides that may be used include AGAGGGTDEGIYDVPLL (the amino acid sequence of which is set forth in SEQ ID NO:35) and AGAGGPQDIYDVPPVR (the amino acid sequence of which is set forth in SEQ ID NO:36).

Another FRET-based assay which can be used for prescreening to identify test compounds that alter the tyrosine kinase activity of Axl is the amplified luminescent proximity homogeneous assay (also known as “AlphaScreen®,” PerkinElmer, Boston, Mass.). In this assay, an Axl peptide is coupled to a first donor population of beads, and used to identify interacting molecules within a population coupled to a second acceptor population of beads. Axl peptides for use in such an assay include peptides corresponding to the ATP and/or substrate binding sites in the intracellular kinase domain. More specifically, Axl peptides include DCLDGLYALMSRC (SEQ ID NO:16) and KKIYNGDYYRQG (SEQ ID NO:17). Other peptides interacting with Axl can be also used for screening assays.

### Assays for Musculoskeletal Activity and Expression of Axl

The methods of the invention provide identification of compounds which modulate the musculoskeletal activity or expression of Axl. Assays for the musculoskeletal activity of Axl are described above and include, but are not limited to, assays which measure alkaline phosphatase activity, assays which measure osteocalcin gene expression, assays which measure bone mineralization, and skeletal phenotyping assays, which characterize bone mass, including bone mineral density, as well as the microarchitecture and biomechanical properties of bone.

Assays for Axl expression are well known in the art and include detecting expression of Axl mRNA and/or protein. Axl mRNA expression may be determined by examining total mRNA expression in cells by transcription profiling using DNA microarrays. The DNA microarray contains expressed sequence tags, deoxyoligonucleotides, or PCR products derived from known or predicted genes. (see, e.g., Bowtell, Nature Genet. Supp. 21:25-32 (1999)). The expression of Axl mRNA may also be determined by Northern blotting. Axl mRNA expression can also be determined by fluorescence-based real-time reverse transcription PCR (RT-PCR). RT-PCR products can be detected by SYBR® Green, TaqMan®, or molecular beacons. Additional strategies for detecting and quantifying mRNA transcript levels via real-time RT-PCR are well known to persons having ordinary skill in the art (see, e.g., Bustin, J. Mol. Endocrinol. 29:23-39 (2002)).

Axl protein levels can be measured in a number of ways. Cells or tissues are harvested from culture or a living organism at a variety of time points following treatment with a test compound and cell lysates are prepared. Levels of Axl protein can then be assessed by SDS-PAGE followed by staining with Coomassie Blue or silver nitrate. Levels of Axl protein may also be assessed by Western blot analysis using an antibody specific for Axl. Protein levels can be measured by using any one of a number of functional assays, including a sandwich or competitive ELISA, or other cell-based assays well known to one of ordinary skill in the art.

### Pharmaceutical Compositions and Methods of Administration

Methods of administering pharmaceutical compositions are known in the art. “Administration” is not limited to any particular delivery system and may include, without limitation, parenteral (including subcutaneous, intravenous, intramedullary, intraarticular, intramuscular, intracavity, or intraperitoneal injection) rectal, topical, transdermal, or oral (for example, in capsules, suspensions, or tablets). Administration to an individual may occur in a single dose or in continuous or intermittent repeat administrations, and in any of a variety of physiologically acceptable salt forms, and/or with an acceptable pharmaceutical carrier and/or additive as part of a pharmaceutical composition (described earlier).

Modulators of Axl may be formulated as pharmaceutical compositions. Physiologically acceptable salt forms and standard pharmaceutical formulation techniques and excipients are well known to persons skilled in the art (see, e.g., Physicians' Desk Reference (PDR) 2003, 57th ed., Medical Economics Company, (2002); and Remington: The Science and Practice of Pharmacy, eds. Gennado et al., 20th ed, Lippincott, Williams & Wilkins, (2000)).

Modulators useful in the methods of the invention may be administered at a dosage from about 1 μg/kg to about 20 mg/kg, depending on the severity of the symptoms and the progression of the disease. The appropriate effective dose is selected by a treating clinician from the following ranges: about 1 μg/kg to about 20 mg/kg, about 1 μg/kg to about 10 mg/kg, about 1 μg/kg to about 1 mg/kg, about 10 μg/kg to about 1 mg/kg, about 10 μg/kg to about 100 μg/kg, about 100 μg to about 1 mg/kg, and about 500 μg/kg to about 1 mg/kg, for example.

Compositions used in the methods of the invention further comprise a pharmaceutically acceptable excipient. As used herein, the phrase “pharmaceutically acceptable excipient” refers to any and all solvents, dispersion media, coatings, antibacterial and antifungal agents, isotonic and absorption delaying agents, and the like, that are compatible with pharmaceutical administration. The use of such media and agents for pharmaceutically active substances are well known in the art. The compositions may also contain other active compounds providing supplemental, additional, or enhanced therapeutic functions. The pharmaceutical compositions may also be included in a container, pack, or dispenser together with instructions for administration.

A pharmaceutical composition is formulated to be compatible with its intended route of administration. Examples of such compositions include crystalline protein formulations, provided naked or in combination with biodegradable polymers (e.g., PEG, PLGA).

As used herein, “modulators” include inhibitors and activators. Included in the methods of the invention are modulators of Axl gene expression and modulators of Axl protein activity. Inhibitors of Axl gene expression are those that inhibit transcription and/or translation of the Axl gene. Inhibitors of Axl protein activity are those that inhibit, e.g., Axl membrane binding, Axl kinase activity, and/or binding of Axl protein to a ligand.

A modulator of the invention may be administered as a pharmaceutical composition in conjunction with carrier gels, matrices, excipients, or other compositions used for guided bone regeneration and/or bone substitution. Examples of such matrices include synthetic polyethylene glycol (PEG)-, hydroxyapatite, collagen and fibrin-based matrices, tisseel fibrin glue, etc. Excipients can include pharmaceutically acceptable salts, polysaccharides, peptides, proteins, amino acids, synthetic polymers, natural polymers, and surfactants.

The Axl modulators are formulated for delivery as injectable or implantable compositions. The composition can be in the form of a cylindrical rod suitable for injecting or implanting in solid state into a body. The injectable formulation includes the inhibitor and a hyaluronic acid ester, as described in detail in U.S. Patent Publication No. 20050287135, which is hereby incorporated by reference. For example, Hyaff11p65 can be used as the hyaluronic acid. The injectable formulation includes the modulator and a calcium phosphate material, such as amorphous apatitic calcium phosphate, poorly crystalline apatitic calcium phosphate, hydroxyapatite, tricalcium phosphate, fluorapatite and combinations thereof, as described in detail in U.S. Patent Publication No. 20050089579, which is hereby incorporated by reference.

Inhibitors of Axl may be co-administered with one or more osteogenic proteins, including, but not limited to, BMP2, BMP-4, BMP-5, BMP-6, BMP-7, BMP-9, BMP-10, BMP-12, BMP-13, MP52, or heterodimers thereof.

An Axl inhibitor may be administered in combination or concomitantly with other therapeutic compounds such as, e.g., bisphosphonate (nitrogen-containing and non-nitrogen-containing), apomine, testosterone, estrogen, sodium fluoride, strontium ranelate, vitamin D and its analogs, calcitonin, calcium supplements, selective estrogen receptor modulators (SERMs, e.g., raloxifene), osteogenic proteins (e.g., BMP2), statins, RANKL inhibitors, cathespin K inhibitors, Wnt pathway modulators e.g. sclerostin antibody, Activators of Non-Genotropic Estrogen-Like Signaling (ANGELS), and parathyroid hormone (PTH). (Apomine is a novel 1,1 bisphosphonate ester, which activates farneion X activated receptor and accelerates degradation of HMG CoA reductase (3-hydroxy-3-methylglutaryl-coenzyme A reductase (see, e.g., U.S. Patent Publication No. 20030036537 and references cited therein). Inhibitors of Axl are co-administered with a bisphosphonate, including but not limited to alendronate, cimadronate, clodronate, EB-1053, etidronates, ibandronate, neridronate, olpadronate, pamidronate, risedronate, tiludronate, YH 529, zolendronate, and pharmaceutically acceptable salts, esters, acids, and mixtures thereof.

Administration of a therapeutic to an individual in accordance with the methods of the invention may also be by means of gene therapy, wherein a nucleic acid sequence encoding the modulator is administered to the patient in vivo or to cells in vitro, which are then introduced into a patient. For specific gene therapy protocols, see Morgan, Gene Therapy Protocols, 2nd ed., Humana Press (2000).

### Methods of Screening and Diagnosis

The present invention can be used to identify subjects who are genetically predisposed to having altered bone density or presently have altered bone density. To screen for and/or diagnose altered bone density, the levels of Axl in a test sample from the subject and a control sample are compared. The presence of an altered level of Axl in the test sample is indicative of an altered bone density and/or a predisposition to developing an altered bone density in the subject. The present invention provides a method for detecting the presence of an Axl variant nucleic acid sequence in a nucleic acid-containing sample, compared to a subject having a wild-type nucleic acid sequence.

The level of Axl in a subject is elevated relative to a control sample, and the subject has decreased bone density or an increased risk of developing decreased bone density. The level of Axl in a subject is decreased relative to a control sample, and the subject has increased bone density or an increased likelihood of developing increased bone density. Anti-Axl specific antibodies or anti-Axl variant specific antibodies can be used to determine the level of the respective proteins in a sample. The invention provides a method for detecting Axl or variants thereof in a subject to be screened or diagnosed which includes contacting an anti-Axl antibody with a cell or protein and detecting binding to the antibody. The antibody can be directly labeled with a compound or detectable label which allows detection of binding to its antigen. Different labels and methods of labeling are known to those of ordinary skill in the art. Examples of the types of labels which can be used in the present invention include enzymes, radioisotopes, fluorescent compounds, colloidal metals, chemiluminescent compounds, phosphorescent compounds, and bioluminescent compounds. The level of Axl can be detected in samples isolated from biological fluids and tissues. Any specimen containing a detectable amount of antigen can be used. A sample in the methods of the invention is bone tissue. The level of Axl gene expression or protein activity in a test sample from a subject can be compared with the level of Axl gene expression or protein activity in a normal cell to determine whether the subject is predisposed to altered bone density.

The antibodies of the invention are suited for use, for example, in immunoassays, including liquid phase or bound to a solid phase carrier. Immunoassays which use antibodies include competitive and non-competitive immunoassays in either a direct or indirect format, such as radioimmunoassays (RIA) and sandwich (immunometric) assays. Antibodies can also be used to detect Axl using immunohistochemical assays on physiological samples.

The present invention provides a method for detecting the presence of an Axl variant nucleic acid sequence in a nucleic acid-containing test sample isolated from a subject, as compared to a control sample having a wild-type nucleic acid sequence. An Axl “variant” as used herein, includes variant Axl nucleic acids and variant Axl proteins. An Axl variant nucleic acid refers to any Axl nucleic acid sequence which does not correspond to the wild-type Axl nucleic acid sequence. An Axl variant protein refers to any Axl amino acid sequence which does not correspond to the wild-type Axl amino acid sequence. The methods of the invention include variants of segments of Axl which do not share sequence identity with the corresponding segment of the wild-type Axl sequence. Variants useful in the methods and assays of the invention include alterations generated by a mutation, a restriction fragment length polymorphism, a single nucleotide polymorphism (SNP), a nucleic acid deletion, or a nucleic acid substitution naturally occurring or intentionally manipulated. Variants also include peptides, or full length proteins, that contain substitutions, deletions, or insertions into the protein backbone, that would still leave a 70% homology to the original protein over the corresponding portion.

The term “isolated polynucleotides” as used herein includes polynucleotides substantially free of other nucleic acids, proteins, lipids, carbohydrates or other materials with which it is naturally associated. Polynucleotide sequences of the invention include DNA and RNA sequences which encode Axl variants. It is understood that all polynucleotides encoding all or a portion of Axl variants are also included herein, such as naturally occurring, synthetic, and intentionally manipulated polynucleotides. The polynucleotides useful in the methods and assays of the invention include sequences that are degenerate as a result of the genetic code. A complementary sequence may include an antisense nucleotide. Also included are fragments (portions) of the above-described nucleic acid sequences that are at least 10-15 bases in length, which is sufficient to permit the fragment to specifically hybridize to DNA of the variant nucleic acid.

Nucleic acid sequences useful in the methods and assays of the invention can be obtained by any method known in the art. For example, DNA can be isolated by: hybridization of genomic or cDNA libraries with probes to detect homologous nucleotide sequences, polymerase chain reaction (PCR) on genomic DNA or cDNA using primers capable of annealing to the DNA sequence of interest, or antibody screening of expression libraries to detect cloned DNA fragments with shared structural features.

The development of specific DNA sequences encoding Axl, or variants thereof, can also be obtained by: isolation of double-stranded DNA sequences from the genomic DNA; chemical manufacture of a DNA sequence to provide the necessary codons for the polypeptide of interest; or in vitro synthesis of a double stranded DNA sequence by reverse transcription of mRNA isolated from a eukaryotic donor cell. In the latter case, a double-stranded DNA complement of mRNA is formed, referred to as cDNA.

The present invention provides any one or more methods for identifying nucleic acid variants associated with altered bone density by detecting the presence of a target Axl variant nucleic acid sequence in sample isolated from a subject having altered bone density as compared to a subject having normal bone density and a wild-type Axl nucleic acid sequence.

The present invention includes methods for identifying allelic variants in a subject. The subject may be homozygous or heterozygous for an Axl variant. As used herein, an “allele” is a gene or nucleotide sequence, such as a single nucleotide polymorphism (SNP), present in more than one form (different sequence) in a genome. “Homozygous,” according to the present invention, indicates that the two copies of the gene or SNP are identical in sequence to the other allele. For example, a subject homozygous for the wild-type Axl gene contains at least two copies of the Axl wild-type sequence. Such a subject would not be predisposed to an altered bone density.

“Heterozygous,” as used herein, indicates that two different copies of the allele are present in the genome, for example one copy of the wild-type allele and one copy of the variant allele. “Heterozygous” also encompasses a subject having two different mutations in its Axl alleles.

The invention provides methods for developing an allelic profile of a subject for an Axl gene. “Allelic profile,” as used herein, is a determination of the composition of a subject's genome in regard to the presence or absence, and the copy number, of the Axl allele or variants thereof.

The invention provides a method of determining predisposition of a subject to altered bone density. The method includes determining the Axl allelic profile of a subject by isolating the nucleic acid specimen from the subject which includes the Axl sequence and determining the presence or absence of a mutation in the Axl nucleic acid sequence. The invention also provides a diagnostic or prognostic method for determining the Axl allelic profile of a subject including isolating a nucleic acid sample from the subject and amplifying the nucleic acid with primers that hybridize to target sequences.

Any method which detects allelic variants can be used. For example, allele specific oligonucleotides (ASOs) can be used as probes to identify such variants. ASO probes can be any length suitable for detecting the sequence of interest. Preferably such probes are 10-50 nucleotides in length and will be detectably labeled by isotopic or nonisotopic methods. The target sequences can be optionally amplified and separated by gel electrophoresis prior to immobilization by Southern blotting. Alternatively, extracts containing unamplified nucleic acid can be transferred to nitrocellulose and probed directly as dot blots.

In addition, allele-specific alterations can be identified by coincidental restriction site alteration. Mutations sometimes alter restriction enzyme cleavage sites or, alternatively, introduce restriction sites were none had previously existed. The change or addition of a restriction enzyme recognition site can be used to identify a particular variant.

Primers used in the methods of the invention include oligonucleotides of sufficient length and appropriate sequence to provide specific initiation of polymerization of a significant number of nucleic acid molecules containing the target nucleic acid under the conditions of stringency for the reaction utilizing the primers. In this manner, it is possible to selectively amplify the specific target nucleic acid sequence containing the nucleic acid of interest. Specifically, the term “primer,” as used herein, refers to a sequence comprising two or more deoxyribonucleotides or ribonucleotides. The primer may be about at least eight nucleotides, which sequence is capable of initiating synthesis of a primer extension product that is substantially complementary to a target nucleic acid strand. The oligonucleotide primer typically contains 15-22 or more nucleotides, although it may contain fewer nucleotides as long as the primer is of sufficient specificity to allow essentially only the amplification of the specifically desired target nucleotide sequence (i.e., the primer is substantially complementary).

Primers used according to the method of the invention are designed to be “substantially” complementary to each strand of mutant nucleotide sequence to be amplified. Substantially complementary means that the primers must be sufficiently complementary to hybridize with their respective strands under conditions which allow the agent for polymerization to function. In other words, the primers should be sufficiently complementary with the flanking sequences to hybridize therewith and permit amplification of the mutant nucleotide sequence. Preferably, the 3′ terminus of the primer that is extended has perfectly base pairing with the complementary flanking strand.

Oligonucleotide primers can be used in any amplification process that produces increased quantities of target nucleic acid, including polymerase chain reaction. Typically, one primer is complementary to the negative (−) strand of the mutant nucleotide sequence and the other is complementary to the positive (+) strand. Annealing the primers to denatured nucleic acid followed by extension with an enzyme, such as the large fragment of DNA Polymerase I (Klenow) or Taq DNA polymerase and nucleotides or ligases, results in newly synthesized + and − strands containing the target nucleic acid. Because these newly synthesized nucleic acids are also templates, repeated cycles of denaturing, primer annealing, and extension results in exponential production of the region (i.e., the target mutant nucleotide sequence) defined by the primer. The product of the amplification reaction is a discrete nucleic acid duplex with termini corresponding to the ends of the specific primers employed. Those of skill in the art will know of other amplification methodologies which can also be utilized to increase the copy number of target nucleic acid.

The nucleic acid from any tissue specimen, in purified or nonpurified form, can be utilized as the starting nucleic acid or acids, provided it contains, or is suspected of containing, the specific nucleic acid sequence containing the target nucleic acid. Thus, the process may employ, for example, DNA or RNA, including messenger RNA (mRNA), wherein DNA or RNA may be single stranded or double stranded. In the event that RNA is to be used as a template, enzymes, and/or conditions optimal for reverse transcribing the template to DNA would be utilized. In addition, a DNA-RNA hybrid which contains one strand of each may be utilized. A mixture of nucleic acids may also be employed, or the nucleic acids produced in a previous amplification reaction herein, using the same or different primers may be so utilized. The mutant nucleotide sequence to be amplified may be a fraction of a larger molecule or can be present initially as a discrete molecule, such that the specific sequence constitutes the entire nucleic acid. It is not necessary that the sequence to be amplified be present initially in a pure form; it may be a minor fraction of a complex mixture, such as contained in whole human or animal DNA.

The amplified product may be detected by Southern blot analysis, without using radioactive probes. In such a process, for example, a small sample of DNA containing a very low level of mutant nucleotide sequence is amplified, and analyzed via a Southern blotting technique. The use of non-radioactive probes or labels is facilitated by the high level of the amplified signal.

Where the target nucleic acid is not amplified, detection using an appropriate hybridization probe may be performed directly on the separated nucleic acid. In those instances where the target nucleic acid is amplified, detection with the appropriate hybridization probe would be performed after amplification.

The probes of the present invention can be used for examining the distribution of the specific fragments detected, as well as the quantitative (relative) degree of binding of the probe for determining the occurrence of specific strongly binding (hybridizing) sequences. The probes of the invention can be detectably labeled with an atom or inorganic radical, most commonly using radionuclides, but also heavy metals can be used. Any radioactive label may be employed which provides for an adequate signal and has sufficient half-life. Other labels include ligands, which can serve as a specific binding pair member for a labeled ligand, and the like. A wide variety of labels routinely employed in immunoassays can be used. Fluorescent compounds include fluorescein and its derivatives, rhodamine and its derivatives, dansyl, umbelliferone, and so forth. Chemiluminescers include luciferin, and 2,3-dihydrophtha-lazinediones (e.g., luminol).

Nucleic acids having an Axl variant detected by the methods of the invention can be further evaluated, detected, cloned, sequenced, and the like, either in solution or after binding to a solid support, by any method usually applied to the detection of a specific DNA sequence such as PCR, oligomer restriction (Saiki, et al., Bio/Technology, 3:1008-1012, 1985), allele-specific oligonucleotide (ASO) probe analysis (Conner, et al., Proc. Natl. Acad. Sci. USA, 80:278, 1983), oligonucleotide ligation assays (OLAs) (Landegren, et al., Science, 241:1077, 1988), and the like.

The present invention provides kits for detecting altered levels or variances in Axl. Such a kit may comprise a probe which is or can be detectably labeled. Such a probe may be an antibody or nucleotide specific for a target protein, or fragments thereof, or a target nucleic acid, or fragment thereof, respectively, wherein the target is indicative, or correlates with, the presence of Axl, or variants thereof. For example, oligonucleotide probes of the present invention can be included in a kit and used for examining the presence of Axl variants, as well as the quantitative (relative) degree of binding of the probe for determining the occurrence of specific strongly binding (hybridizing) sequences, thus indicating the likelihood for an subject having or predisposed to having altered bone density.

The present invention provides a kit that utilizes nucleic acid hybridization to detect the target nucleic acid. The kit may also have containers containing nucleotide(s) for amplification of the target nucleic acid sequence. When it is desirable to amplify the target nucleic acid sequence, such as a variant nucleic acid sequence, this can be accomplished using oligonucleotide(s) that are primers for amplification.

The kit provides a container containing antibodies which bind to a target protein, or fragments thereof, or variants of such a protein, or fragments thereof. Thus, a kit may contain antibodies which bind to wild-type Axl or their variants. Such antibodies can be used to distinguish the presence of a particular Axl variant or the level of expression of such variants in a specimen.

As used in the specification and the appended claims, the singular forms “a,” “an” and “the” include reference to the plural unless the context clearly dictates otherwise. Thus, for example, reference to “an antibody” includes a plurality of such compositions, i.e., “antibodies.”

The following examples provide illustrative embodiments of the invention. One of ordinary skill in the art will recognize the numerous modifications and variations that may be performed without altering the spirit or scope of the present invention. Such modifications and variations are encompassed within the scope of the invention. The Examples do not in any way limit the invention.

### EXAMPLES

### Example 1

### BMP2 Downregulates Axl

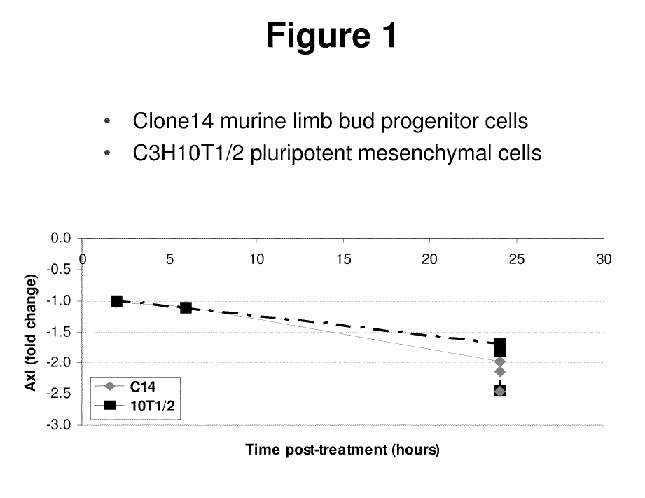

The effect of the osteoinductive factor BMP2 on Axl gene expression was assayed in two murine mesenchymal cell lines, C3H10T½pluripotent mesenchymal cells and Clone 14 murine limb bud progenitor cells. Briefly, C3H10T½ or Clone 14 cells were incubated in the presence of either 100 ng/ml or 320 ng/ml rhBMP2. Samples were obtained at 2, 6, and 24 hours post addition of rhBMP2 and the amount of Axl measured using quantitative RT-PCR. As shown in FIG. 1, Axl expression was significantly downregulated by approximately two-fold within 24 hours of treatment in both C3H10T½ and Clone 14 murine mesenchymal cell lines. C14: Clone 14 cells; 10T½: C3H10T½ cells. These results indicate that Axl is regulated by BMP2.

### Example 2

### Axl siRNA Reduces Axl mRNA Levels

The role of Axl in osteoblast differentiation and activity was investigated using RNA gene expression knockdown techniques in Clone 14 and MC3T3-E1 murine cell lines. As described in detail next, the results of these experiments indicate Axl knockdown both promotes osteoblast differentiation from osteoprogenitor cells and enhances osteoblast function.

The role of Axl in osteoblast differentiation from an osteoprogenitor cell was investigated using RNA interference in the murine Clone 14 cell line. In these experiments, siRNA reagents against murine Axl were transfected into cells treated with either 0 ng/ml or 100 ng/ml BMP2. The induction of osteocalcin gene expression, an established osteoblast marker, was measured to assess osteoblast differentiation 4 days after the onset of treatment and transfection.

siRNA Sequences

siRNAs were purchased from Dharmacon (LaFayette, Co) and have the following sequences:

| | | |
|---|---|---|
| Name | Sequence | SEQ ID NO. |

| | | |
|---|---|---|
| Axl 1 | 5′-GGAAAGAGGUGAACUGGUAUU-3′ | SEQ ID NO: 3 |
| Axl 2 | 5′-CAAGAUGAAUGGAAAGUUGUU-3′ | SEQ ID NO: 4 |
| Axl 3 | 5′-GGAACUGCAUGCUGAAUGAUU-3′ | SEQ ID NO: 5 |
| Axl 4 | 5′-GGAAGAAGGAGACUCGAUAUU-3′ | SEQ ID NO: 6 |
| “Axl | combination of Axl1, 2, 3 | n/a |
| pool” | and 4 | |
| Scramble | 5′GGUAGCUAUUCAGUUACUG-3′ | SEQ ID NO: 7 |
| Runx2/ | 5′-CGUGAAUGGUCAUAAUAACU-3′ | SEQ ID NO: 8 |
| Cbfa1 | | |

Detection of Axl mRNA Levels

Clone 14 cells were seeded at 20,000 cells/well in a 96-well plate and cultured in DMEM media supplemented with 10% fetal bovine serum (FBS) and 1% L-glutamine. The following day, cells were transfected for 4 hours with siRNA at a final concentration of 20 nM using 0.5% (final) Lipofectamine2000 (Invitrogen, Carlsbad, Calif.). Cells were then cultured in DMEM supplemented with 1% FBS and 1% penicillin/streptomycin. Some samples received media supplemented with 100 ng/ml rhBMP2. As negative controls, cells were either mock transfected (no siRNA) or transfected with a non-specific, scrambled, siRNA sequence (NSP).

The knockdown of Axl mRNA was monitored by real-time RT-PCR 24 hours post siRNA transfection. The specificity of the Axl RNAi reagents was confirmed by also monitoring the transcripts of the two most closely related Axl family members: Mer and Tyro3. Media was removed and cells were washed in PBS. Total RNA was purified using the Promega (Madison, Wis.) SV96 Total RNA kit (catalog No. Z3505). RNA was eluted in a final volume of 100 μl. Real-time RT-PCR was performed using 5 μl of RNA per 25 μl reaction in 1×QRT-PCR mastermix (Eurogenetec, Philadelphia, Pa.; catalog No. VWR81002-530). Primers and probes were purchased from Applied Biosystems (ABI, Foster City, Calif.) and used at a final concentration of 1×: Axl (Assay-on-Demand Mm00437221_m1), Mer (Assay-on-Demand Mm0043492_m1), and, Tyro3 (Assay-on-Demand Mm0044547_m1). Gene expression was monitored relative to the housekeeping gene GAPDH (ABI cat #4308310; 200 nM final concentration of probe; 100 nM final concentration of primers).

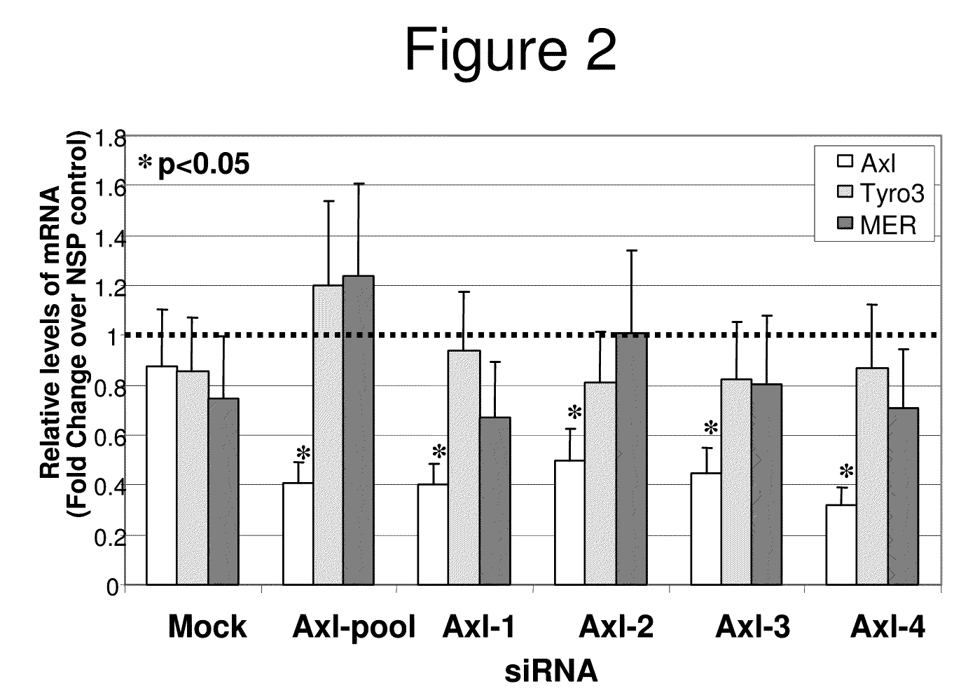

Relative gene expression levels of Axl, Mer and Tyro3 following Axl siRNA knockdown are shown in FIG. 2. The graph shows the relative levels of Axl, Tyro3, and Mer mRNA detected in Clone 14 cells transfected with Axl-specific siRNAs. The data is normalized to the expression levels detected in cells transfected with a scrambled, non-specific siRNA (NSP). The graph shows the relative mRNA levels in cells were transfected without mRNA (Mock), with individual Axl-specific siRNAs (Axl-1, Axl-2, Axl-3, or Axl-4), or with a mixture of the four Axl-specific siRNAs (Axl-pool). The columns are the mean values, and the bars indicate plus and minus the standard deviation. The asterisk (*) indicates that the chances of the observed difference from control being due to a chance alone less than 0.05, i.e. less than 1 in 20. All data is presented as fold change relative to expression levels detected in cells transfected with the non-specific, scrambled siRNA where the level has been set to 1 (dotted line, FIG. 2). In the negative control, mock transfected cells, there was no change in gene expression of any of the three monitored genes. All four Axl siRNA reagents as well as the pool showed a significant reduction in Axl mRNA levels confirming the efficacy of the siRNAs (p<0.05 by t-test; FIG. 2A). Furthermore, all four Axl siRNA reagents as well as the pool showed specificity for targeting Axl transcripts, but gene expression levels for the closely related genes Tyro3 and Mer were not affected following Axl siRNA transfection. These data demonstrate that the siRNA reagents are capable of specifically knocking down Axl mRNA levels in the Clone 14 cells.

### Example 3

### Axl siRNA Increases Osteocalcin Expression

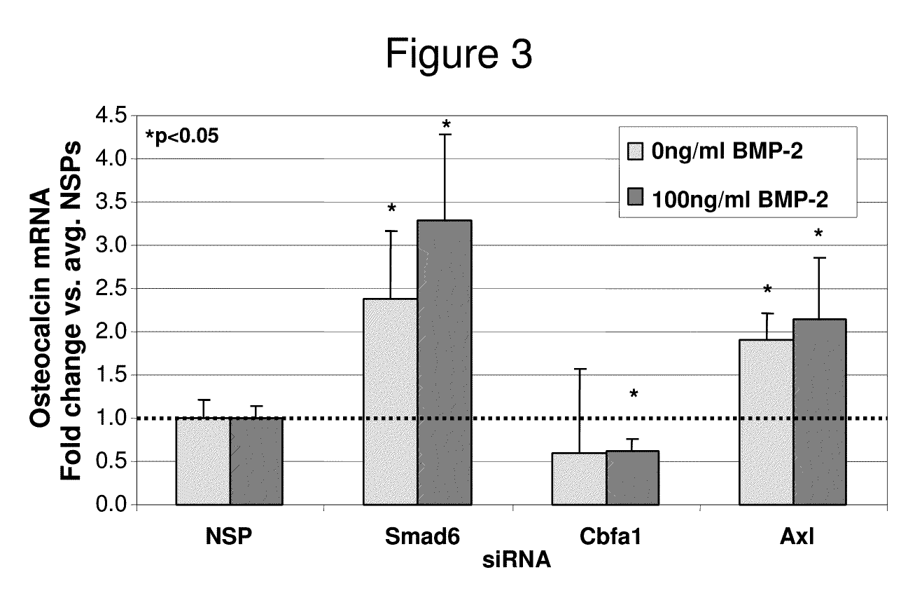

To assess the consequence of Axl knockdown on osteoblast differentiation, osteocalcin mRNA levels were monitored. Osteocalcin is an established marker of late osteoblast differentiation. In this study, the pool of Axl siRNAs described above was transfected into Clone 14 cells and cultured for 4 days in the presence (100 ng/ml) or absence of exogenous rhBMP2, exactly as described above. As positive control for the assay, a pool of siRNAs against Smad6, a known negative regulator of BMP2 signaling, was used (Smad6 in FIG. 3). As a negative control, an siRNA against Runx2/Cbfa1, a known positive regulator of osteoblast differentiation, was used. The sequence of this siRNA is set forth in SEQ ID NO:8 and is shown below:

### 5′-CGUGAAUGGUCAUAAUAACU-3′

An additional negative control was the same non-specific, scrambled siRNA described above (NSP). Osteocalcin mRNA levels were monitored by real-time RT-PCR as above using the primers set forth in SEQ ID NO:9, SEQ ID NO:10, and SEQ ID NO:11 and shown below:

| | |
|---|---|
| 5′-CGGCCCTGAGTCTGACAAA-3′; | (SEQ ID NO:9) |
| 5′-GCCGGAGTCTGTTCACTACCTT-3′; | (SEQ ID NO:10) |
| 5′-CCTTCATGTCCAAGCAGGAGGGCA-3′ | (SEQ ID NO:11) |

FIG. 3 shows the osteocalcin mRNA fold change in the presence and absence of exogenous BMP2 in Clone 14 cells. Osteocalcin mRNA levels are shown relative to those monitored in cells transfected with a scrambled, non-specific siRNA (NSP). Cells were incubated in either the absence (left, lighter bars) or presence (right, darker bars) of 100 ng/ml BMP2. Shown are relative osteocalcin mRNA levels in cells transfected with a scrambled, non-specific siRNA (NSP), with a pool of Smad6-specific siRNA (Smad6), with a Runx2/Cbfa1-specific siRNA (Cbfa1), or with a pool of Axl-specific siRNA (Axl). The columns are the mean values, and the bars indicate plus and minus the standard deviation. The asterisk (*) indicates a probability of less than 0.05. Values are mean+/−SD; * p<0.05.

As predicted, knockdown of Smad6 stimulated osteocalcin expression over 2-fold in the absence of exogenous BMP2 and over 3-fold in the presence of BMP2 (p<0.05; FIG. 3). Conversely, knockdown of Runx2/Cbfa1 dramatically repressed osteoblast differentiation as monitored by osteocalcin mRNA expression in all tested conditions (p<0.05; FIG. 3). Knockdown of Axl expression increases osteocalcin levels after BMP2 stimulation by over 2-fold compared to a non-specific, scramble siRNA, which was used as a control (p<0.05; FIG. 3). Furthermore, in the absence of exogenous BMP2 stimulation, knockdown of Axl results in a 2-fold induction of osteocalcin mRNA (p<0.05; FIG. 3). These data show that inhibition of Axl promotes osteogenic differentiation and that such inhibition can also potentiate the known osteogenic effects of BMP2.

### Example 4

### Axl siRNA Increases Alkaline Phosphatase Activity

Clone 14 cells were seeded at 20,000 cells/well in a 96-well plate and cultured in DMEM media supplemented with 10% FBS and 1% L-glutamine. The following day, cells were transfected for 4 hours with siRNA (SEQ ID NO. 3, 4, 5, or 6) (Dharmacon, Lafayette, Colo.) at a final concentration of 20 nM using 0.5% (final) Lipofectamine2000 (Invitrogen, Carlsbad, Calif.). Cells were then cultured in DMEM supplemented with 1% FBS and 1% penicillin/streptomycin. Some samples received media supplemented with 100 ng/ml rhBMP2. As negative controls, cells were either mock transfected (no siRNA) or transfected with a non-specific, scrambled, siRNA having the nucleotide sequence set forth in SEQ ID NO:7: GGUAGCUAUUCAGUUACUG (SEQ ID NO. 7). The functional consequence of Axl knockdown on osteoblast differentiation was assessed by Alkaline Phosphatase activity. After 4 days of treatment of cells, the media from the wells was aspirated and washed twice with PBS (200 μl/well). 100 μl of distilled water was added per well. Plates were freeze/thawed twice to disrupt and lyse the cells. 50 μl of the lysed cells were added to 50 μl of ALP buffer mix. For 10 ml ALP buffer: 0.1M Glycine, pH 10.3, 16 mg MgCl₂, 80 μl 12.5% Triton X-100, 42 mg p-nitrophenyl phosphate). Reactions were incubated for 30 minutes at 37° C., and stopped by adding 100 μl 0.2 M NaOH to each well. The colormetric reactions were read at 405 nm on a microplate reader.

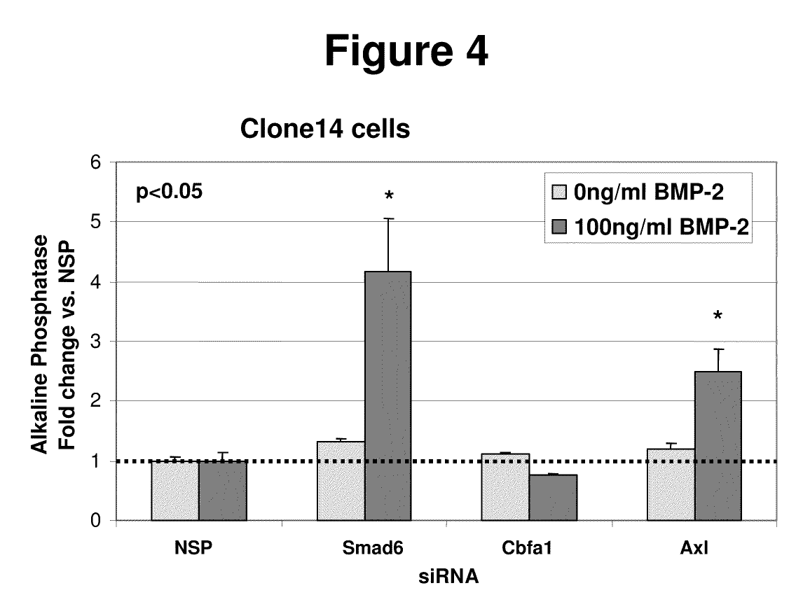

FIG. 4 is a graph that shows Axl knockdown induces alkaline phosphatase activity and that this effect is enhanced by incubation in the presence of BMP2 protein. The graph shows relative alkaline phosphatase activity in cells incubated either in the absence (lighter bars) or presence of 100 ng/ml BMP2 protein (darker bars). The results are shown relative to activity in cells transfected with a scrambled, non-specific siRNA (NSP). Shown are relative levels of alkaline phosphatase in cells transfected with a pool of Smad6-specific siRNA (Smad6), with a Runx2/Cbfa1-specific siRNA (Cbfa1), or with a pool of Axl-specific siRNA (Axl). The columns are the mean values, and the bars indicate plus and minus the standard deviation. The asterisk (*) indicates a probability of less than 0.05. Values are mean+/−SD; p<0.05.

### Example 5

### Overexpression of Axl Represses Osteocalcin Expression

Clone 14 cells were seeded at 20,000 cells/well in a 96-well plate and cultured in DMEM media supplemented with 10% FBS and 1% L-glutamine. The following day, cells were transfected for 4 hours with 100 ng/well of each plasmid using 0.5% (final) Lipofectamine2000 (Invitrogen, Carlsbad, Calif.). Cells were then cultured in DMEM supplemented with 1% FBS and 1% pen/strep. Some samples received media supplemented with 100 ng/ml rhBMP2. RNA was isolated 4 days following transfection and osteocalcin mRNA was measured by real-time RT-PCR and the primers set forth in SEQ ID NO:9, SEQ ID NO:10, and SEQ ID NO:11 and shown above. Overexpression of Axl protein was demonstrated by Western blot analyses.

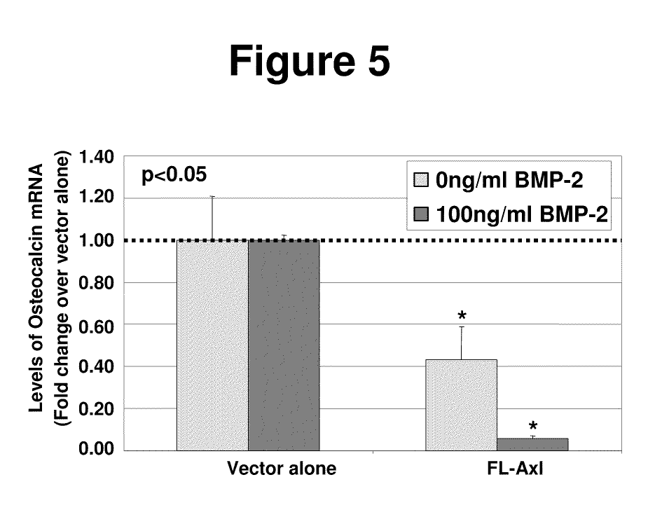

FIG. 5 shows that Axl overexpression represses osteocalcin mRNA levels. FIG. 5 is a graph that shows osteocalcin mRNA levels in cells incubated either in the absence (lighter bars) or presence of 100 ng/ml BMP2 protein (darker bars). The results are shown relative to those monitored in cells transfected with a non-specific vector. Vector alone: cells transfected with a non-specific vector; FL-Axl: cells transfected with a vector expressing a full length Axl. The columns are the mean values, and the bars indicate plus and minus the standard deviation. The asterisk (*) indicates a probability of less than 0.05. Values are mean+/−SD; * p<0.05.

### Example 6

### Axl siRNA Promotes Formation of Mineralized Nodules

Further experiments evaluated osteoblast activity as indicated by the formation of mineralized nodules in vitro after transient knockdown of Axl expression using RNAi technology.

MC3T3-E1 cells were seeded at a density of 50,000 cells/well of a 6-well plate and cultured in Alpha media supplemented with 1% glutamine and 10% FBS. The following day the cells were transfected with 20 nM (final concentration) siRNA using 0.4% (final) Oligofectamine (Invitrogen) for 4 hours. The siRNAs used in this study include the pool of Axl siRNAs and the Cbfa1/Runx2 siRNA described above. In addition two control treatments were included: a mock transfection where no siRNA was introduced and a non-specific, scrambled siRNA (as described).

Following transfection, the cells were maintained in Alpha media supplemented with 1% glutamine, 10% FBS and 10 mM β-glycerophosphate, a cofactor necessary for mineralization, with no other osteogenic agents. The media was changed every 3 days and the assay stopped on Day 17 post-transfection.

To determine mineralization, cells were washed in PBS, fixed in cold ethanol and stained with Alizarin red using standard protocols. Semi-quantification of the extent of mineralization was conducted by de-staining the cells in 10% Cetylpyridinium chloride in 10 mM sodium phosphate for 15 minutes at room temperature. Supernatants were removed and absorbance at 570 nm measured. Concentrations were determined by obtaining absorbance measurements of a standard curve of known dilutions of Alizarin red (range from 50-400 uM). Mineralization was visually assessed by the presence of red-stained nodules.

Axl knockdown resulted in a qualitative increase in the extent of mineralization when compared to either the nonspecific, scrambled siRNA or mock transfected cells. Semi-quantification of alizarin red staining indicated that Axl knockdown resulted in an approximately 20% increase in formation of mineralized nodules compared to the controls. In contrast, knockdown of the negative control Runx2/Cbfa1 showed a dramatic reduction in mineralization. These data suggest that Axl inhibition can potentiate and possibly generate BMP2-like osteogenic differentiation.

### Example 7

### Calvarial Organ Culture Assay

An ex vivo calvarial organ culture model was used to evaluate the response of osteoblasts in a physiological bone microenvironment to Axl inhibition. A soluble mAxl extracellular domain/Fc chimera (Axl/FC, R&D Systems, Minneapolis, Minn.) was used as an inhibitor as it would disrupt ligand binding. Calvaria from 4-day old neonatal ICR mice were dissected and cut into two pieces along the sagittal suture. After incubation overnight in serum-free BGJ media+0.1% BSA, calvariae were incubated with 0.1 μg/ml Axl/Fc for 1, 2, 4 or 7 days or remained untreated (Control) in BGJ media+1% FCS. Axl/Fc was removed from culture medium after the prescribed length of time, and calvaria were incubated with BGJ media+1% FCS for the remainder of the 7 day culture period. Hemi-calvariae from 4 individual mice were used for each experimental condition. Calvariae were then fixed in 10% neutral phosphate buffered formaldehyde, embedded in paraffin, sectioned at 4 μm, and stained with hematoxylin and eosin. Total bone area and number of osteoblasts were quantified using histomorphometric techniques.

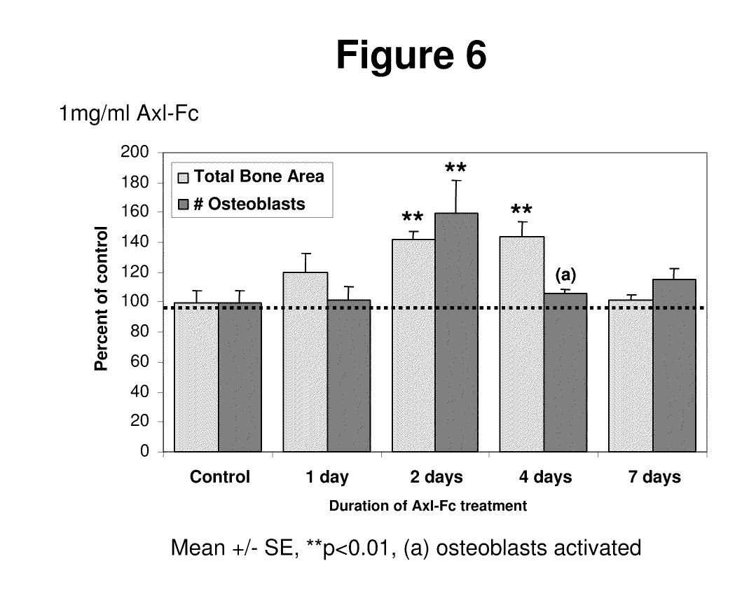

As shown in FIG. 6, brief exposure to Axl/Fc for 2 days resulted in a significant increase in number of osteoblasts and total bone area. Similarly, Axl/Fc treatment for 4 days increased total bone area, but osteoblast number was equivalent to control cultures. At both timepoints, osteoblasts appeared to be activated as evidenced by their plump, cuboidal morphology. In summary, inhibition of Axl promoted osteoblast activity and formation of new bone in this ex vivo calvaria model in a manner similar to treatment with BMP2. The graph shows relative total bone area (left bars), and number of osteoblasts (right bars) Values are mean+/−SE, ** p<0.01, asterisks (**) above the bar indicates that osteoblasts are activated.

As predicted, knockdown of Smad6 stimulated osteocalcin expression over 2-fold in the absence of exogenous BMP2 and over 3-fold in the presence of BMP2 (p<0.05; FIG. 3). Conversely, knockdown of Runx2/Cbfa1 dramatically repressed osteoblast differentiation as monitored by osteocalcin mRNA expression in all tested conditions (p<0.05; FIG. 3). Knockdown of Axl expression increases osteocalcin levels after BMP2 stimulation by over 2-fold compared to a non-specific, scramble siRNA, which was used as a control (p<0.05; FIG. 3). Furthermore, in the absence of exogenous BMP2 stimulation, knockdown of Axl results in a 2-fold induction of osteocalcin mRNA (p<0.05; FIG. 3). These data show inhibition of Axl promotes osteogenic differentiation and that such inhibition can also potentiate the known osteogenic effects of BMP2.

### Example 8

### Axl “Kinase-Dead” does not Repress Osteocalcin Expression

A “kinase dead” Axl mutant (K576R) was developed which replaces a conserved lysine in the ATP binding pocket and results in inactivation of kinase activity. This permits evaluation of the role of Axl's kinase activity in osteoblast biology.

To confirm expression and investigate kinase activity of the “kinase-dead” mutant, 293A cells were plated at a density of 5×10⁶ cells in 10 cm dishes. Cells were incubated overnight and transfected using Lipofectamine™ 2000 (Invitrogen, Carlsbad, Calif.) with 12 μg of plasmid of interest for 20 minutes. A media change was performed and cells were incubated for another 48 hours to permit protein expression. Cells were lysed and protein determined by bicinchoninic acid (BCA) assay (Pierce Protein Research Products, Rockford, Ill.). Western Breeze Chemiluminescent Immunodetection (Invitrogen) was used to determine whether or not there is kinase activity in “kinase dead” Axl. Cell lysates were incubated with an Axl antibody obtained from Cell Signaling (Danvers, Mass., catalog #4977) at a 1:50 dilution and incubated with an Anti-V5 and AntiV5-HRP antibody (Invitrogen) and a Phospho-Tyrosine Mouse monoclonal antibody (Cell Signaling catalog #9411) in 5% with BSA, 1×TBS, 0.1% Tween®-20 at 4° C. overnight.

To investigate whether or not Axl “kinase-dead” suppressed osteocalcin expression, Clone 14 cells (mouse osteoblasts) were used. Clone 14 cells were maintained in Dulbecco's modified Eagle's medium containing 10% FBS (Atlanta Biologicals, Lawrenceville, Ga.) at 37° C. in humidified 10% CO₂ in air. For all treatments, cells were plated in six-well culture plates at a density of 3×10⁶ cells/well and incubated overnight. Cells were then washed with PBS and transfected (Lipofectamine™ 2000) with 4 μg of either Axl DNA, Axl “kinase-dead” DNA or control DNA. After 3 hours, media was changed and 100 ng/ml of BMP2 was added. One day later, total RNA was isolated using a RNeasy kit (Qiagen, Valencia, Calif.) and treated with DNase I (1 unit/5 μg of RNA) at room temperature for 30 minutes. mRNA for osteoblast marker genes was detected by real-time PCR using an ABI Prism 7000 sequence detection system (Applied Biosystems) and then normalized to GAPDH levels.

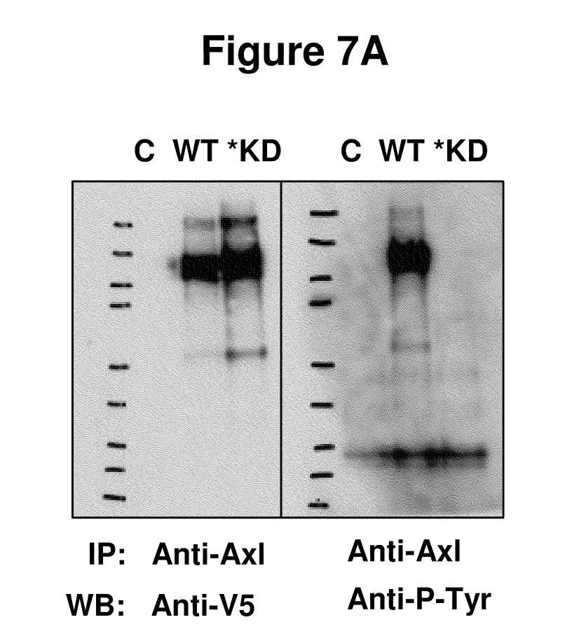

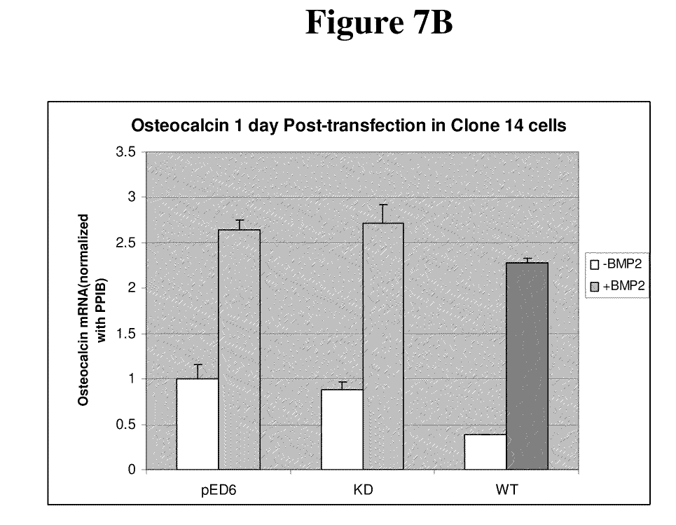

As shown in FIG. 7(A) 293A cells transfected with either wild-type (WT) Axl or Axl “Kinase-dead” (KD) and then lysed and immunoprecipitated with anti-Axl antibody before being probed with anti-P-Tyr antibody on a Western blot demonstrated that, in contrast to WT Axl, Axl KD has no ability to phosphorylate itself. In addition, as shown in FIG. 7 (B), transfection of Clone 14 osteoblasts with the WT Axl results in a reduction in osteocalcin expression whereas transfection of Axl KD has no such effect, showing that the kinase activity of Axl is required for Axl's effects on osteoblast differentiation.

### Example 9

### Axl Knockout Mice have Increased Bone Mass

Axl knockout mice (“t1453 Axl”, referred to herein as KO) were obtained from Deltagen (San Mateo, Calif.). Axl KO and age matched Wild-type (WT) mice were evaluated at 26 weeks for skeletal phenotype.

Excised femur was analyzed using peripheral quantitative computed tomography (pQCT). One 0.5-mm PQCT slice obtained 2.5 mm proximal from the distal end was used to compute total and trabecular density and another 0.5 mm slice obtained 9 mm proximal from the distal end (in the mid-shaft region) was used to analyze cortical density for the femoral metaphysis. Total, trabecular and cortical volumetric bone densities of distal femur of Axl-KO and age-matched WT mice groups (n=8-10/group) were compared using Student's t-test.

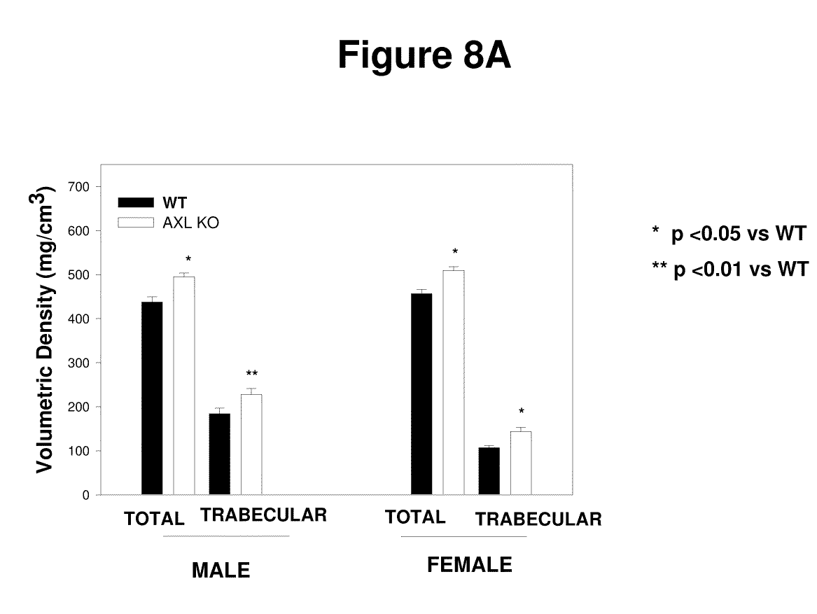

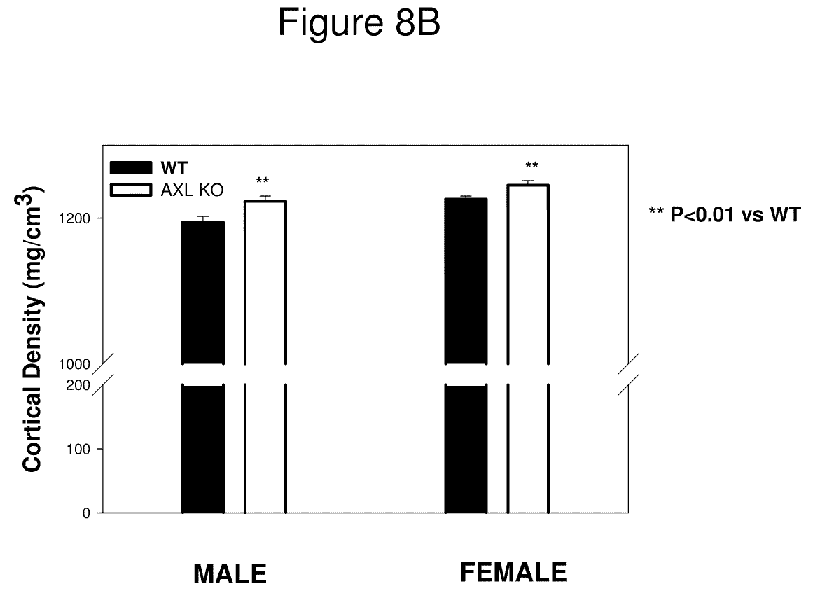

FIG. 8 shows that 26-week-old Axl KO male and female mice have a high bone mass phenotype as revealed by PQCT measurements of the volumetric bone mineral density (vBMD) of the distal femur. FIG. 8A shows that, compared to wild type, male mice had a 13% increase in total vBMD and a 23% increase in trabecular vBMD; while female mice had an 11% increase in total vBMD and a 34% increase in trabecular vBMD. FIG. 8B shows that, compared to wild type, male mice had a 1.55% increase in cortical vBMD, while female mice had a 2.40% increase in vBMD.

The specification is most thoroughly understood in light of the teachings of the references cited within the specification. The embodiments within the specification provide an illustration of embodiments of the invention and should not be construed to limit the scope of the invention. The skilled artisan readily recognizes that many other embodiments are encompassed by the invention. All publications, patents, and biological sequences cited in this disclosure are incorporated by reference in their entirety. To the extent the material incorporated by reference contradicts or is inconsistent with the present specification, the present specification will supersede any such material. The citation of any references herein is not an admission that such references are prior art to the present invention.

What is claimed is:

1. A method of treating or preventing a bone disorder in a mammal, the method comprising administering to the mammal an inhibitor of Axl gene expression or Axl protein activity, wherein the inhibitor is not bone morphogenetic protein 2 (BMP2) protein.

2. The method according to claim 1 wherein the inhibitor inhibits Axl protein activity.

3. The method according to claim 2 wherein the Axl protein activity is kinase activity.

4. The method of claim 1 wherein the inhibitor has the structural formula (I): 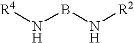
or a salt, hydrate, solvate or N-oxide thereof, wherein:
B is 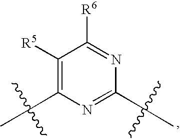
wherein R⁵ and R⁶ together form a saturated or unsaturated alkylene or saturated or unsaturated heteroalkylene chain of 3 to 4 atoms, optionally substituted with one or more R^{a} and/or R^{b};
R² is selected from the group consisting of (C₆-C₂₀) aryl optionally substituted with one or more R⁸, a 5-20 membered heteroaryl optionally substituted with one or more R⁸, a (C₇-C₂₈) arylalkyl optionally substituted with one or more R⁸ and a 6-28 membered heteroarylalkyl optionally substituted with one or more R⁸;
R⁴ is a saturated or unsaturated, bridged or unbridged cycloalkyl containing a total of from 3 to 16 annular carbon atoms that is substituted with an R⁷ group, with the proviso that when R⁴ is an unsaturated unbridged cycloalkyl, or a saturated bridged cycloalkyl, this R⁷ substituent is optional, wherein R⁴ is further optionally substituted with one or more R^{f};
R⁷ is selected from the group consisting of —C(O)OR^{d}, —C(O)NR^{d}R^{d}, —C(O)NR^{d}OR^{d}, or —C(O)NR^{d}NR^{d}R^{d};
each R⁸ group is, independently of the others, selected from the group consisting of a water-solubilizing group, R^{a}, R^{b}, C₁-C₈, alkyl optionally substituted with one or more R^{a} and/or R^{b}, C₃-C₈ cycloalkyl optionally substituted with one or more R^{a} and/or R^{b}, heterocycloalkyl containing 3 to 12 annular atoms, optionally substituted with one or more R^{a} and/or R^{b}, C₁-C₈ alkoxy optionally substituted with one or more R^{a} and/or R^{b}, and —O—(CH₂)ₓ—R^{b}, where x is 1-6;
each R^{a} is, independently of the others, selected from the group consisting of hydrogen, C₁-C₈ alkyl, bridged or unbridged C₃-C₁₀ cycloalkyl, bridged or unbridged heterocycloalkyl containing 3 to 12 annular atoms, heteroaryl, (C₆-C₁₄) aryl, and (C₇-C₂₀) arylalkyl, wherein R^{a} is optionally substituted with one or more R^{f};
each R^{b} is, independently of the others, a suitable group selected from the group consisting of ═O, —OR^{a}, (C₁-C₃) haloalkyloxy, ═S, —SR^{a}, ═NR^{a}, ═NOR^{a}, —NR^{c}R^{c}, halogen, —C₁-C₃ haloalkyl, —CN, —NC, —OCN, —SCN, —NO, —NO₂, ═N₂, —N₃, —S(O)R^{a}, —S(O)₂R^{a}, —S(O)₂OR^{a}, —S(O)NR^{c}R^{c}, —S(O)₂NR^{c}R^{c}, —OS(O)R^{a}, —OS(O)₂R^{a}, —OS(O)₂OR^{a}, —OS(O)₂NR^{c}R^{c}, —C(O)R^{a}, —C(O)OR^{a}, —C(O)NR^{c}R^{c}, —C(O)NR^{a}OR^{a}, —C(NH)NR^{c}R^{c}, —C(NR^{a})NR^{c}R^{c}, —C(NOH)R^{a}, —C(NOH)NR^{c}R^{c}, —OC(O)R^{a}, —OC(O)OR^{a}, —OC(O)NR^{c}R^{c}, —OC(NH)NR^{c}R^{c} and —OC(NR^{a})NR^{c}R^{c};
each R^{c} is, independently of the others, is R^{a} or two R^{c} that are bonded to the same nitrogen atom taken together with the nitrogen atom to which they are both attached form a heterocycloalkyl group containing 5 to 8 annular atoms, which optionally includes from 1 to 3 additional heteroatomic groups selected from the group consisting of —O—, —S—, —N(—(CH₂)_{y}—R^{a})—, —N(—(CH₂)_{y}—C(O)R^{a})—, —N(—(CH₂)_{y}—C(O)OR^{a})—, —N(—(CH₂)_{y}—S(O)₂R^{a})—, —N(—(CH₂)_{y}—S(O)₂OR^{a})— and —N(—(CH₂)_{y}—C(O)NR^{a}R^{a})— where y is 0-6, wherein the heterocycloalkyl is optionally substituted with one or more R^{f};
each R^{d} is, independently of the others, selected from the group consisting of R^{a}, R^{c} and a chiral auxiliary group; and
each R^{f} is independently —C₁-C₈ alkoxy, —C₁-C₈ alkyl, —C₁-C₆ haloalkyl, cyano, nitro, amino, (C₁-C₈ alkyl)amino, di(C₁-C₈ alkyl)amino, phenyl, benzyl, oxo, or halogen,
or any two R^{f} bonded to adjacent atoms, taken together with the atoms to which they are each attached, form a fused saturated or unsaturated cycloalkyl or a fused saturated or unsaturated heterocycloalkyl group containing 5 to 8 annular atoms, wherein the formed cycloalkyl and heterocycloalkyl groups are optionally substituted with one or more groups which are each independently selected from halogen, C₁-C₈ alkyl, and phenyl.

5. The method according to claim 1, wherein the bone disorder is osteopenia, osteomalacia, osteoporosis, osteoarthritis, osteomyeloma, osteodystrophy, Paget's disease, osteogenesis imperfecta, bone sclerosis, aplastic bone disorder, humoral hypercalcemic myeloma, multiple myeloma, or bone thinning following metastasis.

6. The method according to claim 5, wherein the disorder is osteoporosis.

7. The method according to claim 6, wherein the osteoporosis is post-menopausal, steroid-induced, senile, or thyroxin-use induced.

8. The method according to claim 1, wherein the bone disorder is caused by at least one of hypercalcemia, chronic renal disease, kidney dialysis, primary hyperparathyroidism, secondary hyperparathyroidism, inflammatory bowel disease, Crohn's disease, long-term use of corticosteroids, or long-term use of gonadotropin releasing hormone (GnRH) agonists or antagonists.

9. The method according to claim 1, wherein the treatment increases osteoblast number or osteoblast activity.

10. The method according to claim 9 wherein increased osteoblast number or activity results in an increase in expression of an osteoblast marker.

11. The method according to claim 10 wherein the osteoblast marker is osteocalcin, alkaline phosphatase, or collagen type I.

12. The method according to claim 9 wherein the increased osteoblast number or osteoblast activity reduces at least one of: the level of bone deterioration, the loss of bone mass, the loss of bone mineral density, the degeneration of bone quality, or the degeneration of bone microstructural integrity.

13. The method according to claim 9 wherein the inhibitor is a compound, a protein, a peptide, an antibody, an aptamer, or a polynucleotide.

14. The method according to claim 13, wherein the inhibitor prevents or reduces Axl gene transcription.

15. The method according to claim 13, wherein the inhibitor prevents or reduces translation of Axl messenger ribonucleic acid (mRNA).

16. The method according to claim 15 wherein the inhibitor is a polynucleotide.

17. The method according to claim 16 wherein the polynucleotide is ribonucleic acid (RNA).

18. The method according to claim 17 wherein the RNA is antisense.

19. The method according to claim 17 wherein the RNA is double stranded RNA.

20. The method according to claim 19 wherein the RNA is short interfering RNA (siRNA).

21. The method according to claim 20 wherein the siRNA is about 15 to about 40 nucleotides in length.

22. The method according to claim 21 wherein the siRNA nucleotide sequence is SEQ ID NO:3, SEQ ID NO:4, SEQ ID NO:5, or SEQ ID NO:6.

23. The method according to claim 20 wherein the siRNA comprises the sequence of a micro RNA (miRNA).

24. The method according to claim 16 wherein the polynucleotide is deoxyribonucleic acid (DNA).

25. The method according to claim 24 wherein the DNA is antisense DNA.

26. The method according to claim 1, wherein the inhibitor decreases the tyrosine kinase activity of Axl protein.

27. The method according to claim 10, wherein the inhibitor inhibits interaction between Axl protein and at least one Axl protein ligand.

28. The method according to claim 27 wherein the inhibitor inhibits interaction between Axl protein and at least one of growth arrest-specific 6 (Gas6) protein; protein S; p85α, subunit of phosphatidylinositol 3-kinase (PI3K) protein, p85β subunit of PI3K protein; phospholipase C-γ (PLC-γ) protein, growth factor receptor-bound protein 2 (Grb2); c-Src protein; Ras protein; Akt protein; ERK/MAPK protein; NF-κB protein; GSK3 protein; IL-15 receptor α subunit protein; or mTOR protein.

29. The method according to claim 28 wherein the inhibitor prevents activation of Axl protein by Gas6 protein.

30. The method of claim 29 wherein the inhibitor binds to the Gas6 major binding site of the Axl protein.

31. The method of claim 29 wherein the inhibitor prevents binding of Gas6 to Axl.

32. The method according to claim 13 wherein the inhibitor is a protein.

33. The method according to claim 32 wherein the protein is a protease.

34. The method according to claim 32 wherein the protein is a soluble Axl protein or a fragment thereof, a mutant Axl protein or a fragment thereof, an Axl protein ligand or a fragment thereof.

35. The method according to claim 32 wherein the protein is a mutant Axl protein.

36. The method according to claim 35 wherein the mutant Axl protein has a substitution of arginine for lysine at amino acid position 567 of SEQ ID NO:2.

37. The method according to claim 13, wherein the inhibitor is an antibody.

38. The method according to claim 32 wherein the inhibitor is a small modular immunopharmaceutical (SMIP).

39. The method according to claim 37, wherein the antibody is a human antibody or a humanized antibody.

40. The method according to claim 37, wherein the antibody specifically binds to Axl protein.

41. The method according to claim 37 wherein the antibody binds to the Gas6 major binding site of the Axl protein.

42. The method according to claim 37, wherein the antibody specifically binds to an Axl protein ligand other than Gas6.

43. The method according to claim 1, wherein the mammal is human.

44. The method according to claim 1, wherein the inhibitor is administered systemically.

45. The method according to claim 1, wherein the inhibitor is administered repeatedly over a period of time of at least two weeks.

46. The method according to claim 1, wherein the inhibitor is administered at the site of injury.

47. The method according to claim 1, further comprising administering to the mammal at least one agent selected from the group consisting of a bisphosphonate, a bone morphogenetic protein (BMP), a calcitonin, an estrogen, a selective estrogen receptor inhibitor, a parathyroid hormone, and a vitamin, a RANKL inhibitor, a Cathepsin K inhibitor, a sclerostin inhibitor, and strontium ranelate.

48. The method according to claim 47, wherein the agent is a bisphosphonate.

49. The method according to claim 47, wherein the agent is a BMP.

50. The method according to claim 49, wherein the BMP is BMP2, BMP4, BMP6, or heterodimers thereof.

51. The method according to claim 50 wherein the BMP is a BMP2/BMP6 heterodimer.

52. A method of identifying a compound that modulates Axl protein kinase activity comprising:
a) providing an Axl polypeptide having kinase activity;
b) providing a substrate which is phosphorylated in the presence of the Axl polypeptide;
c) mixing the Axl polypeptide and the substrate under conditions which allow phosphorylation of the substrate;
d) contacting the mixture in c) with a compound; and
e) determining whether or not the compound
modulates Axl kinase activity.

53. The method according to claim 52 wherein the Axl polypeptide comprises the amino acid sequence of SEQ ID NO:13, SEQ ID NO:37, SEQ ID NO:38, SEQ ID NO:39, SEQ ID NO:40, SEQ ID NO:41, or SEQ ID NO:42.

54. The method according to claim 52 wherein the Axl polypeptide comprises the amino acid sequence of SEQ ID NO:13, SEQ ID NO:37, SEQ ID NO:38, SEQ ID NO:39, SEQ ID NO:40, SEQ ID NO:41, SEQ ID NO:42, or SEQ ID NO:43.

55. The method according to claim 52 wherein the substrate comprises the amino acid sequence of SEQ ID NO:16, SEQ ID NO:17, SEQ ID NO:35, or SEQ ID NO:36

56. A method of screening for altered bone density in a subject comprising:
a) obtaining a test sample from the subject;
b) determining the level of Axl gene expression or the level of Axl protein activity in the test sample;
c) comparing the level of Axl gene expression or the level of Axl protein activity in the test sample to the level Axl gene expression or the level of Axl protein activity in a control sample,
wherein an altered level of Axl gene expression or altered level of Axl protein activity in the test sample relative to the level of Axl gene expression or the level of Axl protein activity in the control sample is indicative of an altered bone density.

57. The method according to claim 56, wherein the level of Axl gene expression or the level of Axl protein activity in the test sample is increased relative to the control sample.

58. The method according to claim 56, wherein the level of Axl gene expression or the level of Axl protein activity in the test sample is decreased relative to the control sample.

59. The method according to claim 56, wherein the level of Axl protein activity is determined using a capture reagent that specifically binds Axl protein.

60. The method according to claim 59, wherein the Axl capture reagent is an antibody.

61. The method according to claim 60, wherein the antibody is detected using a detectable label.

62. The method according to claim 61, wherein the detectable label is a radioisotope, a fluorescent compound, a bioluminescent compound, a colorimetric compound, or a chemiluminescent compound.

63. A kit comprising a capture reagent that specifically binds at least one Axl polypeptide, buffer, and reagents for detecting binding of the capture reagent to at least one Axl polypeptide.

64. The kit according to claim 63 wherein the capture reagent comprises a detectable label.

65. The kit according to claim 63 wherein the capture reagent is an antibody.

66. A method of screening for altered level of bone mineral density, altered bone mass, altered bone quality, altered bone formation, or altered bone microstructural integrity in a subject comprising determining the presence of at least one mutation in a polynucleotide encoding Axl in a test sample from the subject, wherein the presence of said at least one mutation in a polynucleotide encoding Axl is indicative of an altered bone density, altered bone mass, altered bone quality, or altered bone formation in the subject.

67. The method according to claim 66, wherein the presence or the absence of at least one mutation in a polynucleotide encoding Axl is detected by contacting the sample with an oligonucleotide probe that hybridizes specifically with a polynucleotide encoding Axl.

68. The method according to claim 67, wherein the oligonucleotide probe comprises at least about 15 nucleotides of a polynucleotide encoding an Axl polypeptide.

69. The method according to claim 66, wherein the polynucleotide is selected from the group consisting of DNA, genomic DNA, complementary DNA (cDNA), RNA, and mRNA.

70. The method according to claim 69, wherein the polynucleotide encodes a mutant Axl protein.

71. The method according to claim 70 wherein the mutant Axl protein has a substitution of arginine for lysine at amino acid position 567 of SEQ ID NO:2.

72. A polynucleotide comprising a nucleotide sequence selected from SEQ ID NO:3, SEQ ID NO:4, SEQ ID NO:5, and SEQ ID NO:6.
